(19) United States
(12) Patent Application Publication
(10) Pub. No.: US 2020/0283920 A1
(43) Pub. Date: Sep. 10, 2020

(54) PROCESS FOR ENRICHMENT OF THE CO2 CONCENTRATION IN THE CO2-STREAMS FROM THE POST-COMBUSTION AND PRE-COMBUSTION STATIONARY SOURCES OF CO2 EMISSION UPSTREAM OF FURTHER PROCESSING

(71) Applicant: Faramarz Bairamijamal, Gaithersburg, MD (US)

(72) Inventor: Faramarz Bairamijamal, Gaithersburg, MD (US)

(21) Appl. No.: 16/820,610

(22) Filed: Mar. 16, 2020

Related U.S. Application Data
(63) Continuation of application No. 14/392,066, filed on Aug. 5, 2015.

(51) Int. Cl. C25B 9/10; C25B 1/10; C25B 1/12; B01D 53/00; F22B 1/00; F01K 7/22; F01K 25/10; F01K 13/00; C25B 9/08; C01B 3/12; C01C 1/04; C05C 1/00; C07C 29/151; C07C 41/01; C10G 2/00; C10L 1/04; C10L 3/08; C25B 1/00; F02C 3/04; F02C 3/20

(52) U.S. Cl. CPC C25B 9/10; C10L 2290/42; C25B 1/12; B01D 53/002; F22B 1/003; F01K 7/22; F01K 25/103; F01K 13/00; C25B 9/08; C01B 3/12; C01C 1/04; C05C 1/00; C07C 29/1518; C07C 41/01; C10G 2/30; C10L 1/04; C10L 3/08; C25B 1/00; F02C 3/04; F02C 3/20; B01D 2258/0233; B01D 2258/0283; B01D 2257/504; B01D 2259/65; F23J 2900/15061; B01D 2256/22; B01D 2258/025; Y02P 20/131; Y02P 20/129; Y02P 20/152; Y02P 20/57; Y02E 50/32; Y02P 20/124; Y02P 20/13; Y02E 20/16; Y02E 20/326; Y02E 60/366; Y02E 50/12; Y02C 10/12; C01B 2203/0283; C10L 2200/0492; C10L 2290/38; C25B 1/10

(57) ABSTRACT

The present process invention in continuation to the U.S. Ser. No. 14/392,066 appertains to Advanced Combustion in post-combustion carbon capture, wherein the CO₂-containing flue gas, said CO2-Stream, is cleaned from harmful constituents, recirculated, oxygenized and employed for combustion for the fossil fuels, referred to Flue Gas Oxy-Fueling in order to obtain a CO₂-rich gas upstream to CO2-CC with significantly less gas flow rate subject to further processing. This continuation process patent also presents processing to prepare a CO₂-rich CO2-Stream for the pre-combustion carbon capture downstream of gasification and gas cleaning process; or from the secondary CO2-Stream that stems from the cathodic syngas [CO/2H₂] downstream of HPLTE-SG of patent parent, then downstream of the HP/IP-water shift converters in [CO₂/3H₂] composition, whereas the CO₂-rich CO2-Stream from either pre-combustion process is routed to the CO2-CC for CO₂ cooling and condensation section of the U.S. Ser. No. 14/392,066 to obtain liquid carbon dioxide for re-use as new fossil energy resource.

### CROSS-REFERENCE TO RELATED APPLICATION

The present continuation patent relates to the parent patent U.S. Ser. No. 14/392,066, which has been associated with the PCT/EP2014/000443, and the WO 2014/127913 A3 that refers to the U.S. Provisional Patent Application 61/850,685 with the priority date of Feb. 21, 2013.

The present process invention presents the undisclosed information in connection with the CO2-Stream that is more specified in the upstream processing to the CO2-Stream with respect to the pretreatment of that CO2-Stream in order to derive a CO₂-enriched CO2-Stream for the downstream section CO2-CC of the said patent, i.e. for compression, cooling of CO2-Stream and separation of the carbon dioxide from the CO₂-enriched CO2-Stream to obtain liquid carbon dioxide by condensation of CO₂ in the parent patent.

In connection with the enhancement in the CO₂ concentration of the CO2-Stream, there is also primary and secondary carbon ratio disclosed now that in turn is interlinked with the First Bairamijamal Cycle for waste heat recovery in the CO2-HR and power generation in the CO2-PG, yet more specifically in the closing path of the First Thermodynamic Cycle (the trajectories are depicted in FIGS. 4A, 5A and 5B).

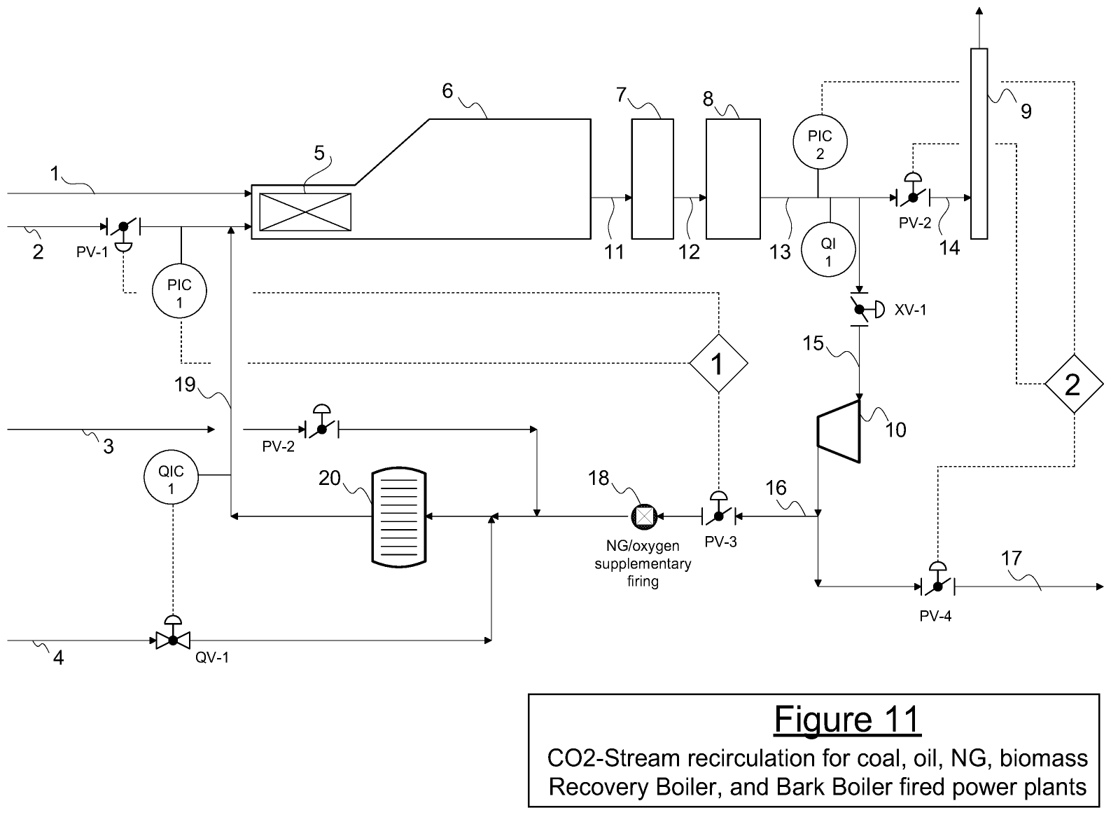

The figures of present patent in continuation are continued in numbering to the parent patent, i.e. FIGS. 11, 12 and 13.

The claims of present patent in continuation are continued in numbering to the parent patent, i.e. with the following claims 61 to 76. The independent claims are 61, 62, 63, 68, 69, 70, 71 and 73. The dependent claims are 64, 65, 66, 67, 72, 74, 75 and 76.

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### THE SUMMARY OF THE INVENTION

The present process invention in continuation to the U.S. Ser. No. 14/392,066 performs the enhancement of CO₂ concentration in the CO2-Stream for both, post-combustion as well as pre-combustion carbon capture, which results to number of advantageous features associated with the processing i.e. lowering the production costs to obtain the liquid carbon dioxide from CO₂-rich gas stream for the compression, cooling, and the CO₂ condensation at one hand, while reduction in the investment for the number and the size of equipment due significantly lower gas flow rate at the other hand (v.i. in the tables in the embodiments with respect to CO₂-constituent of the CO2-Stream upstream of CO2-CC as well as the gas flow rates therein).

The post-combustion embodiment of the present invention presents the Advanced Combustion of fossil fuels that encompasses the pretreatment and CO₂-enrichment of the CO2-Stream for the advantageous carbon-rich fossil power plants from the petcoke in the first priority, followed with most important fuel for this process, the coal in every grade, then crude oil, following with bio-mass, as well as the waste-to-energy, waste-to-hydrocarbons plants and the natural gas in CHP combined heat and power generation plants, yet more importantly for the gas turbine single or combined cycle plants. This process applies also to other CO₂-emitting plants (the Stationary CO₂ Sources) like the Black Liquor firing in the Recovery Boiler of pulp and paper industry. Therefore the present process aims at specifically these two major fossil energy power plants in the first place, i.e. coal power plants and the gas turbine single cycle or combined cycle power plants.

The present process invention inyroduces also the Advanced Combustion, wherein the nitrogen-rich air as combustion gas is no longer employed for combustion of fossil fuels.

The Advanced Combustion for the post-combustion carbon capture embodiment according to the present invention is further distinguished;
(i) by recirculation of the clean flue gas;
(ii) by addition of oxygen (from the HPLTE-SG section of U.S. Ser. No. 14/392,066) into the recirculated flue gas, referred to as flue gas oxy-fueling,
(iii) by preheating of the flue gas-oxygen gas stream; and
(iv) by catalytic oxidation of the residual trace of the carbon monoxide in the flue gas to carbon dioxide before the recirculated oxygenized flue gas is utilized for the combustion of fossil fuel, wherein;
(v) the purge gas from the flue gas recirculation is characterized by high carbon dioxide constituent accompanied with residual oxygen past combustion at saturated water, yet with little traces of carbon monoxide, v.i. for further processing and carbon dioxide capture.

The characteristic thermodynamic features from the flue gas side are:
a) Heat exchange in the scrubber, condensation of combustion water vapor
b) Flue gas recirculation
c) Partial discharge of the flue gas (i.e. discharge of high concentrated CO2-Stream to the CO2-CC section)
d) Addition of oxygen into the recirculated flue gas for combustion of the fuel

Therefore, from the flue gas thermodynamics point of view, the present Advanced Combustion presents with flue gas and flue gas oxy-fueling in the fossil fuel fired power plants a semi-closed, variant of the First Ericson Cycle integrated into the classic Rankin Cycle.

Likewise in the gas turbine plants and from the thermodynamics point of view, the present Advanced Combustion with the semi-closed flue gas and flue gas oxy-fueling, the present patent presents a variant of the First Ericson Cycle integrated into the classic Brayton Cycle.

From the heat recovery side, the thermodynamics of the combination of the classic Rankin Cycle (with steam as the working fluid) together with the First Bairamijamal Cycle (with the carbon dioxide as working fluid) leads to a Dual-Combined Cycle, wherein the heat recovery for power generation reaches out to a gross thermal efficiency in the margin of 65% to 75%.

If the Advanced Combustion is integrated in a new gas turbine power plant (or an existing gas turbine single cycle upgraded to combined cycle), the parent patent's First Bairamijamal Cycle with the working fluid carbon dioxide combined with the balanced heat recovery with the steam as working fluid together in a new heat recovery presents a gas turbine Triple-Combined Cycles with gross thermal efficiency in the margin of 80% to 85% with reference to the most advanced GE 9HA Class gas turbine.

The Advanced Combustion for the pre-combustion embodiment of present invention for primary CO2-Stream from the gasification process and for the secondary syngas of the HPLTE-SG past water-shift converter(s) implies the following six new specific features;
(i) the CO₂-enrichment of the primary lean CO2-Stream
(ii) the CO₂-enrichment of the secondary lean CO2-Stream, i.e. the secondary CO2-Stream that stems from the cathodic [CO/2H₂] first downstream of HPLTE-SG, then over the HP/IP-water shift converters, as described in the parent patent and depicted in the FIG. 3, thus the secondary CO2-Stream in this patent in continuation presents a CO₂-lean gas stream consisting in composition [CO₂/3H₂]
(iii) the separation of carbon dioxide via selexol, rectisol or Benfield from that CO2-Stream, which is obtained either from gasification or from the liquid CO₂-water electroyte in the HPLTE-SG and then in the HP/IP water shift converters, wherein the secondary CO2-Stream consists of CO₂/3H₂ composition with traces of carbon monoxide
(iv) the purification treatment of either CO₂-rich CO2-Streams, i.e. the CO₂-rich gas is purified from water and absorbens specifically for the further treatment in the CO2-CC section
(v) the re-compression of CO-rich rest gas and;
(vi) the recycling of the non-condensable carbon monoxide containing purge gas downstream of CO2-CC back to the main stream of the gasification.

The CO2-Stream from the gasification process comprises the low pressure, intermediate pressure, and most importantly the high pressure gasification of all kind of carbonaciuous feedstock, most significantly coal, petrolium coke, and carbon-rich waste materials as outlined in the U.S. Ser. No. 14/392,066.

### BACKGROUND AND THE DETAILED DESCRIPTION OF THE RELATED ART

The presentation of the state-of-the-art is compiled with reference to U.S. Ser. No. 14/392,066 for the Stationary Sources of CO₂ emission.

The other three presently applied technologies for capture of carbon dioxide and re-use of CO₂ as new fossil energy resource are the ExxonMobil Air Vacuumig in [1] and Carbon Engineering that takes in the air CO₂ for their process [2]. These two latter technologies employ carbon dioxide from the air at the concentration of 0.04 Vol %, thus leading to unimaginable large size of plants if ever feasible for midsize or large scale commercial operation. As Carbon Engineering states, the costs of carbon capture reaches to $100.- per tons of captured carbon dioxide, leading to a predicted production costs of $4.- gasoline a gallon on site. Considering the logistics, distribution and taxes, this might lead to a gasoline price of $5.- to $6.- a gallon while the current gasoline price is posted out at less as $3.- a gallon in North East USA.

The fourth state-of-the-art references to the Carbon Recycling International plant in Island, commonly known as George Olah Plant that utilizes the geothermal carbon dioxide with high CO₂ concentration at ca. 95 Vol %, albeight with extremely low CO₂ stream in terms of mass flow rate available [3]. The George Olah plant demonstrates the production of high end products from CO₂ at high yield and economic feasibility, yet then it faces restrains in terms of commercially viable solution due to very low geothermal gas flow. For instance, the geothermal energy contributes only to ca. 0.2% of US energy consumption in the United States.

Although the current state-of-the-art technologies via [1], [2] and [3] do not take on the major sources of CO₂ emission, all these four processes however, eventually comprise carbon capture and conversion of the captured carbon dioxide to high value products, specifically transportation fuels.

The present patent in continuation refers to the state-of-the-art, as outlined for the pre-combustion carbon capture in the parent patent for the CO2-Stream and presented in FIG. 1, stream 7 from 6 to CO2-CC section's 8, as well as in the block diagrams FIG. 6 and FIG. 7 and FIG. 10 of U.S. Ser. No. 14/392,066.

The present patent in continuation refers also to the state-of-the-art, as distinguished for the post-combustion carbon capture of the Stationary Sources of CO₂ emission in the parent patent for the CO2-Stream U.S. Ser. No. 14/392,066 that has been presented in FIG. 2, stream 6 from 5 to CO2-CC section's compression stage 7, as well as in the block diagrams FIG. 8 and FIG. 9 of U.S. Ser. No. 14/392,066.

The post-combustion carbon capture's primary focus of the present patent in continuation embraces the huge CO₂ emission from the Stationary Sources of CO₂ emission. For instance, the current U.S. energy cosumption of ca. 86.3% stems in one or other way from the fossil energy carbonacuous resources i.e. coal, petrolium, natural gas, biomass, and waste-to-energy.

The post-combustion carbon capture encompasses also the majority of US power generation that is operational from the CO₂ emitting fossil energy. According to EIA report for 2019, 64.8% of electricity is generated by use of fossil resources i.e. from coal, natural gas, petrolium products biomass and municipal waste to energy in the United States [4].

For this reason, the present process invention in continuation presents the embodiment of the invention with reference to the two major kind of fossil power plants without any limitation for other embodiments, i.e. for coal power plant and for the gas turbine power plants, the latter applicable for both for GT single cycle and GT combined cycle.

The current situation in the carbon capture from post-combustion CO2-Streams in U.S. Ser. No. 14/392,066, specifically the flue gas of fossil power plants, is encountered with very low concentration of carbon dioxide in the flue gas, because the current practice of fossil fuel combustion utilizes air as the source of oxygen with only 21 O₂ Vol % for the combustion. The low CO₂ concentration stems predominantly from the nitrogen constituent of air of ca. 78.5 N₂ Vol %. Since the air oxygen lies in the margin of ca. 21 Vol %, the conventional combustion of the fossil fuel in the combustion section is encumbered with the large portion of useless nitrogen. At the other hand, the relatively low concentration of oxygen from the air compels to use more intake air in order to keep an excess oxygen in the flue gas, which in turn promotes a more effective combustion, albeit to more adversse nitrogen to the combustion, ergo more in the flue gas.

The low concentration of the carbon dioxide leads inevitably to very high flue gas flux that leads to more extensive processing in terms of operation costs as well as to larger size of the equipment, particularily expensive compressor, followed by the size of heat exchangers. For instance, the low CO₂ concentration at high gas flow in the U.S. Ser. No. 14/392,066 requires more compressors in number as well as in larger size to deal with.

The condensation of CO₂ out of a dilute CO2-Stream requires also lower operation temperature in the CO2-CC that in turn increases the operation costs significantly.

These disadvantageous points in the U.S. Ser. No. 14/392,066 could be resolved by introduction of Advanced Combustion, which delivers for U.S. Ser. No. 14/392,066 a very high concentrated CO2-rich CO2-Stream at low gas flow rate. Both factors lead to number of extraordinary advantages, both in processing for low operation costs as well in investment of the plants.

The Advanced Combustion according to the present patent in continuation comprises the use of abundant oxygen obtained from the HPLTE-SG now that would had been released utmost into the atmosphere due to huge surplus, way more than the demand could ever absorb.

TABLE 1
Typical flue gas constituents of existing fossil power plants compared
with the flue gas post implementation of Advanced Combustion

| | CO2-Stream to CO2-CC | | | |
|---|---|---|---|---|
| | Current state of flue | | Integration | |
| | gas in conventional | | of Advanced | |
| | combustion (1 | | Combustion (2 | |
| | Flue gas constituents | | | |
| | Coal | Gas | Coal | Gas |
| | power | turbine | power | turbine |
| | plants | plants | plants | plants |
| Nitrogen | 70% | 76% | 0% | 0% |
| Oxygen | 5% | 14% | 8% | 12% |
| Carbon dioxide | 12% | 4% | 91.8% | 79.0% |
| Water moisture | 11% | 5% | 0.11% | 0.05% |
| Nobel gases, as “Argon” | 1% | 1% | 0.05% | 0.87% |
| Carbon monoxide | 0% | 0% | 0.01% | 0.00% |
| Flue gas stream in Nm³/h | 1200000 | 1100000 | 325000 | 64000 |
| Reduction of gas flow in % | — | — | Ca. 27% | Ca. 6% |

1) Pollutants like NOx, SOx, Mercury, Antimuon, Black Carbon, ash and soot in normal mode of operation not considered. These pollutants are removed and disposed of in the scrubber of the parent patent.
2) Refers to Advanced Combustion to the flue gas downstream of the scrubber (e.g. FIG. 11, element 8)

The result of the Advanced Combustion in this patent in continuation is best represented in the tables 2 and 3 to each embodiment at a glance.

Given the 92 Vol % CO₂ concentration in the flue gas of a coal power plant and to 80 Vol % CO₂ in the flue gas of a gas turbine plant the CO2-Stream sets out on comparison with the air 0.041 Vol % CO₂, the present Advanced Combustion presents the capability of more than two thousands times more concentrated CO₂ in the CO2-Stream for the further processing.

The gas flow rate of the CO2-Stream of a coal power plant can be reduced up to 27% of the CO2-Stream obtained from the conventional combustion (vide Table 1 and Stream 17 in the Table 2). The flow rate of CO2-Stream in the gas turbine reaches to around 6% of conventional combustion in flow rate. These results could be inventive resolved for the post-combustion embodiment by the following specific features, which are collectively introduced as Advanced Combustion, i.e.:
1) Recirculation of cleaned flue gas downstream of the flue gas scrubber (FIG. 2, element 5)
2) Adding oxygen, primarily the anodic oxygen available in excess from the HPLTE-SG
3) Routing the recirculated oxygenized clean flue gas back to combustion chamber, hence this referred to as flue gas oxy-fueling in the Advanced Combustion.

As result of (1), (2) and (3), there is:
4) neither nitrogen encumbered in the combustion gas nor present in the flue gas.
5) The discharge of CO₂-rich CO2-Stream in very high concentration can be routed to the CO2-CC section for cooling of the CO2-Stream and separation of CO₂ by way of condensation in significantly more effective way now,
6) a very low gas flow rate of CO₂-rich CO2-Stream for further processing
7) As the technological feasibility of the entire process proffers, all the CO₂ containing side-streams can be integrated readily into the overall process (vide infra CO₂ absorber), thus the concept for Zero-CO2-Emission Fossil Energy is feasibly realized
8) The implementation of the Advanced Combustion in the new plants paves the way for more efficient smaller gas turbine's compressor compartment, smaller compact HRSG and also in the coal power plants with smaller sized combustion chamber and HRSG due the capability for flue gas oxy-fueling at an oxygen over 30 Vol % upstream to the combustion chamber, yet on a pace with excess oxygen in the flue gas.

### FIELD OF THE INVENTION

The field of the present patent in continuation concerns with Advanced Combustion that supports the technological solution to the global warming and Climate Change presented in the parent patent U.S. Ser. No. 14/392,066.

In pursuant to the advancement of Clean Energy, the present patent in continuation comprises the pre-combustion as well post-combustion carbon dioxide capture in all Stationary Sources of CO₂ emission, thus it encompasses the major Green House Gas emitting sources to the degree that other emissions like from vehicle, jet planes, commercial and residential sources can be then readily addressed by the nature soon.

The present patent in continuation supports all other sections of the parent patent, i.e. it comprises the recovery of the useless waste heat released currently into the atmosphere (which heats the globe directly) via operation of CO2-HR section, the conversion of the recovered waste heat to useful energy by operation of the First Bairamijamal Thermodynamic Cycle in CO2-PG, which supplies the utmost portion of the necessary energy for carbon capture in CO2-Stream, and CO2-CC section. Most importantly the First Thermodynamic Cycle generates to the great extent the AC power in the CO2-PG that backs up the HPLTE-SG electrolysis in addition to the multi-stage oxygen and syngas expander turbines after the AC/DC converter (FIG. 3, 17). The CO2-PG generates also another portion of power for dispatch into the grid.

The present patent in continuation also forges ahead in more straight way to the new generation of power plants, referred to in the parent patent as super-efficient hydrogen based fossil power generation with H₂/O₂ torches for Direct Steam generation and re-superheating of steam according to the Second Bairamijamal Cycle described therein.

### DETAILED DESCRIPTION OF THE INVENTION

The embodiment of Advanced Combustion is outlied for each three embodiments individually, because of the distinguishing differences and similarities in each embodiment.

Elaborations and further connections to the sections and embodiments of the parent patent are provided in the section for the abbreviations, special technical expressions and terms at the end of present patent in continuation.

The detail description for each embodiment includes the start up period and transient period, in an existing fossil energy plant, wherein the implementation of the Advanced Combustion and the integration of the parent patent is considered up to new normal mode of operation with the associated process control architecture.

The present invention provides more specific information to the parent patent process invention, to which the CO2-Stream is distinguished with high CO₂ concentration at very low flow rate. The distinctive specifics of this CO2-Stream comprises it's characteristics from the onset of the plant operation, i.e. from the cold start (“the Black Start”) period of the plant, then up to the full load plant capacity, whereas the CO2-Stream undergoes peculiar treatment in the preparation CO₂-rich CO2-Stream upstream to the CO2-CC section, as well as how the CO2-Stream is utilized as a carrier gas for the combustion after oxygen is added to.

This peculiarity of the CO2-Stream in the present process invention is conceived of for the post-combustion purpose in a way, wherein the intake air for combustion is entirely replaced by oxygen-added recirculated CO2-Stream back to the intake and combustion section (referred to flue gas oxy-fueling). By this processing of CO2-Stream, the adverse impacts caused by concommitent nitrogen from the intake air in the existing operational plants in is eliminated totally.

These particular features of the CO2-Stream are presented here for two CO2-Streams in the post-combustion carbon capture and condensation, as well as in one pre-combustion carbon capture and condensation. These three peculiar features of the CO2-Stream are specifically addressed to the existing plants and/or future fossil energy plants, i.e. for:
(I) post-combustion carbon capture applied for the CO2-Stream in the flue gas of fossil fuel fired power generation plants
(II) post-combustion carbon capture applied for the CO2-Stream in the flue gas of gas turbine single cycle and/or flue gas from gas turbine combined cycle
(III) pre-combustion carbon capture from the CO2-Stream obtained from the HPLTE-SG and/or from the gasification process.

The description and points of references in the content of the present process invention to the CO2-Stream are associated with the three embodiments (I), (II) and (III) that is more disclosed in the following three embodiments.

### Embodiment (I): Specifics and Points of Reference in the Parent Patent's Post-Combustion CO2-Stream from the Fossil Fuel Fired Power Plants

In this embodiment, the detailed description of the present process invention is encompassed for the integration of CO2-Stream, pretreatment of the CO2-Stream in the coal, oil, natural gas, biomass fired power plants as well as Black Liquor combustion in the Recovery Boiler and the Bark Boiler firing of the pulp and paper too.

This present embodiment (I) references to the post-combustion carbon capture, which has been previously disclosed in part, inherently in the FIG. 2 equipment 2, 4, 5, 7 (in the first and second stages) with their respective elements, and also to the process streams 1, 3, 6, 40 (down stream of the first stage). The post-combustion carbon capture in the present patent references also to the FIG. 3 with the stream 58.

One of the pertinent block diagrams for this patent in continuation relates to the block diagram in FIG. 8, presented in the boundary limit I for the post-combustion process with the sections and streams 1, 2, 3, 8, 9, 16, 17, 18, 25, as well in part for 27.

The other pertinent block diagram for this more detail disclosure relates to the block diagram in FIG. 9, presented with the boundary limit I for the post-combustion with the process sections and streams 1, 2, 3, 4, 5, 6, 13, 14, 15, 22, 23, including in part for the stream 24.

The embodiment (I) comprises the following six specific features and steps from the cold start of the plant, then up to the normal mode of operation at full load including the intermittent de-sooting procedure, i.e.:
1) The flue gas recirculation to obtain a CO₂-rich CO2-Stream upstream of the CO2-CC section
2) Oxy-fueling of the recirculated flue gas to reduce the nitrogen in the ramp up period; rather than, to eliminate the presence of nitrogen from the intake combustion air in the normal mode of operation totally.
3) Integration of other parent patent's internal oxygen-rich gases into the flue gas recirculation and flue gas oxy-fueling line
4) Implementation of Zero-Pollutants-Emission for removal of all hurmful constituents of coal and other fossil feedstock preventing from reaching to the atmosphere or in the plant's effluent, i.e. those constituents are removed in the multi-stage scrubber and disposed of all harmful constituents as solid waste obtained downstream of waste water treatment
5) Implementation of Zero-CO2-Emission Fossil Energy, which includes also CO2-Streams of little flow rate in the plant, i.e. removal of rest CO₂ from the purge gas downstream of CO2-CC via CO₂-absorber and it's integration in the scrubber stream.
6) The operation of two over-ride process control loops for the gradual transitional mode, from the cold start, then up to the full load operation of the plant.

### BRIEF DESCRIPTION OF THE EMBODIMENT (I) VIA FIG. 11

The Advanced Combustion with the flue gas recirculation and flue gas oxy-fueling for the CO₂ enrichment in post-combustion CO2-Streams of fossil fuel fired power plants upstream of the CO2-CC section is presented by the brief description and FIG. 11 without any limitation to other embodiment(s).

The FIG. 11 shall present a typical power plant that operates currently with any fossil fuel 1, preferably coal, wherein the combustion of 1 is carried out via air's oxygen taken from the atmosphere 2. The typical combustion section of the plant is depicted by 5 with the HRSG 6 and the subsequent flue gas treatment and cleaning 7, wherein the hot flue gas 11 is further cleaned from ash, soot and other aerosol pollutants in the existing electrostatic precipitator, the DeNOₓ, and de-sulforization section DeSOₓ, eventually obtaining the flue gas 12 that fullfils the current regulations for release into the atmosphere via chimney. Under current mode of operation, the stream 12 shall present the CO2-Stream upstream of the present process invention containing typically 10% to 12% CO₂ by volume wet obtained from the combustion.

The CO2-Stream 12, presented as CO2-lean gas under the currently operational plants, is the subject of the present patent in continuation as it pertains to the detail processing for the above mentioned eight specific features in the Advanced Combustion. A typical coal power plant ramps up with ca. 5% to 10% of coal load 1 with nearly full load of intake combustion air 2.

It shall be highlighted that the integration of parent patent and the present patent in continuation implies the existence of a water storage tank according to the water codes for DEMIN water at an existing site as usual. A new smaller size intermediary tank for pretreated DEMIN water according to the applicable standards for the electrolysis as an interim tank is considered, as well as few new high-pressure storage tanks for the liquid CO₂ at the boundary limit of the plant that is required for the implementation of the Advanced Combustion. Hence this, the ramp up of the plant with the integration of parent patent and present patent presumes the operation of HPLTE-SG that is fed by the liquid carbon dioxide and water from those storage tanks with power supply from the grid at the initial phase of start up. Due to this circumstance, the downstream adjacent plant for high-value products can commence the operation with the syngas already from HPLTE-SG, while the necessary oxygen for the start up is supplied from HPLTE-SG via stream 4 to the power plant.

For this reason, the initial stage of the start-up period is carried out with intake air first. Hence this, the hot flue gas 12 with still abundant portion of nitrogen can be cleaned from the residual aerosol, soot, pollutants, NOx and SOx and other harmful constituent in the parent patent's scrubber 8 (depicted in the FIG. 2 as 2 and 5, e.g. a Venturi scrubber and a multi-stage trickle scrubber). In that scrubber 8, the hot CO₂-lean initial flue gas 12 is further cooled down to ca. 85 Degree of Fahrenheit 13, dehydrated on to that temperature at that prevailing pressure, whereas the most water vapor from the combustion is captured and removed from the circulation loops of the scrubber and finally discharged to the waste water treatment section (FIG. 2, stream 32).

The cold end flue gas 13 downstream of the scrubber 8 is utmost recirculated 15, first via the partially OPEN XV-1 and nearly CLOSED PV-2 to the flue gas recirculation blower 10, then routing the flue gas back to the combustion chamber, wherein the recirculated flue gas 16 is preheated by a heat exchanger or rather than by direct firing of natural gas with the portion of oxygen (from the HPLTE-SG) in the supplementary firing 18. The pure oxygen 4 from the HPLTE-SG section is added to the preheated recirculated flue gas before passing through the CO-oxidation reactor 20, thus termed flue gas oxy-fueling with an oxygen content, set to meet preferably 25% to 40% oxygen by volume via QIC-1 upstream of the combustion chamber of the firing 5. Other oxygen containing residual gases 3, most importantly the oxygen-rich CO₂-containing gas downstream of the CO2-CC section, more preferably the re-gasified LOX is also re-routed back to the firing section.

By this way, the initial intake combustion air 2 through PV-1 can be reduced gradually in relative fast pace, while the residual nitrogen is purged by stream 14 through PV-2 to the chimney 9 out of the recirculating flue gas. The first over-ride control circuit via PIC-1, PV-1 and PV-3 directs the transient process control until the smooth transition is completed. The progress can be observed with declining nitrogen via QI-1. By then, the first over-ride control loop CLOSES the PV-1, vide infra.

Depending on the size of HRSG, it takes traditionally two to three more hours by an now increasing coal load from the initial load to 20% coal like in a 500 MW coal power plant until the nitrogen is purged out entirely. After “No Nitrogen” observed by QI-1, the second over-ride control loop takes effect by CLOSING PV-2 and OPENING of PV-4, while the CO2-Stream compressor (FIG. 2, group of equipment 7) runs with 17 at the initial recirculation mode over the by-pass and anti-surge cooler while discharges gradually the CO₂-rich and nearly-dehydrated CO2-Stream with 85% to 90% CO₂ and 10% to 15% O₂ out of the flue gas to the downstream CO2-CC section. The subsequent ramp up of the plant continues up to the full load takes under the regimen of flue gas oxy-fueling that is by then supplied by oxygen from the upcoming liquid carbon dioxide stemming from the carbon constituent of the fossil fuel.

Under the initial start-up and then in normal mode of operation, the residual off gas downstream of the CO2-CC section (i.e. stream 44 downstream of the off gas expander 9 in FIG. 2) consists typically of 92 Vol % oxygen with a residual carbon dioxie of ca. 7 Vol % and residue about 1 Vol % non-condensing inert gases, typically the nitrogen that stems from the nitrogen constituent of the coal. In pursuant to Zero-CO2-Emission Fossil Energy, this offgas is first passed through a CO₂-absorber at the same pressure prevailing in the CO2-CC that removes the CO₂ with water. The discharge of the CO₂ absorber is routed to the scrubber, wherein the residual CO₂ disorbed and routed to the main flue gas stream.

The oxygen rich off gas downstream of the CO₂ absorber at a pressure close to the CO2-CC pressure is chilled down and dehydrated, by then undergoes an isenthalpic expansion, wherefrom a LOX stream is obtained and a GOX stream encumbered with the inert non-condensable (of coal orgin) components are released into the atmosphere. That LOX undergoes a regasification with the available thermal waste energy e.g. in CO2-HR section, and rerouted back to the combustion, figuratively presented with the stream 3.

The non-condensable gases in the GOX (like from the nitrogen constituent of coal) will be purged downstream of CO2-CC section from the oxygen-rich GOX.
FIG. 11 CO2-Stream recirculation for coal, oil, NG, biomass, Recovery Boiler, Bark Boiler fired power plants

### ELEMENTS IN THE FIG. 11

| | |
|---|---|
| 1 | Fossil fuel supply line, stands for coal, oil, NG, biomass as well as |
| | Black Liquor for Recovery Boiler, barks for Bark Boiler of the |
| | pulp and paper. |
| 2 | Intake combustion air for the initial ramp up period |
| 3 | Oxygen stream from the re-gasified LOX from the CO2-CC |
| | section's purge gas downstream of the CO2 absorber that is |
| | integrated in the CO2-HR for regasification and preheating (i.e. the |
| | stream 44 in FIG. 2 or the stream 27) |
| 4 | Oxygen for the flue gas oxy-fueling from the HPLTE-SG, (i.e. |
| | stream 58 in FIG. 3) |
| 5 | Combustion chamber of the power plant |
| 6 | Typically the existing HRSG, the Heat Recovery Steam Generation |
| | section of the power plant |
| 7 | Typically existing section for the flue gas cleaning with DeNOx, |
| | DeSOx, electrostatic precipitator for ash, soot and pollutant |
| | removal upstream of the parent patent |
| 8 | Parent patent's section for the flue gas scrubbing, (i.e. Figure's 2 |
| | equipment 2 and 5, as well as the streams 2, 3, 4, 5, 6 at the flue |
| | gas cold end upstream of the flue gas recirculation and flue gas |
| | compression 7). The scrubber 8 of FIG. 11 comprises also the |
| | discharge of condensed water resulted from the combustion of fuel |
| | and oxygen at the hot end (i.e. FIG. 2, stream 24 and 29, |
| | eventually stream 32 to the waste water treatment) |
| 9 | Chimney |
| 10 | Flue gas recirculation blower (i.e. Figure's 2, element 4 and the 7 |
| | in the first stage), driven e.g. by off gas expander turbine (i.e. |
| | elements 8 and 9 in FIG. 2) or driven by one of the CO₂-turbines |
| 11 | Hot flue gas downstream of HRSG to flue gas DeNOx, DeSOx, |
| | electrostatic precipitator for ash, soot and pollutant removal section |
| 12 | Hot flue gas to the parent patent's flue gas scrubbers (i.e. 2 and 5 |
| | of the FIG. 2) |
| 13 | Cleaned cooled down flue gas downstream of scrubbers |
| 14 | Flue gas to purge during the ramp up period to the atmosphere via |
| | the chimney |
| 15 | Flue gas for recirculation (i.e. stream 6 in FIG. 2) |
| 16 | Pressure elevated cleaned flue gas to recirculation |
| 17 | Pressure elevated CO₂-rich cleaned flue gas with residual oxygen |
| | post the combustion after the ramp-up period (typically with 85% |
| | to 90% by volume CO₂ and ca. 10% by volume excess oxygen), |
| | passed on to the next pressure stage of the flue gas compressor (i.e. |
| | element 7 in FIG. 2) upstream of CO2-CC section |
| 18 | Reheating of the recirculated flue gas upstream of combustion, |
| | preferably by use of natural gas-oxygen torch fed from HPLTE- |
| | SG's oxygen |
| 19 | Recirculated oxygen-enriched pre-heated flue gas downstream of |
| | the oxy-fueling and upstream of the combustion chamber with ca. |
| | 25% to 40% oxygen by volume. |
| 20 | Catalytic oxidation reactor for oxidation of carbon moxide traces to |
| | carbon dioxide in the recirculated flue gas |

### FURTHER ELEMENTS IN FIG. 11

PV-1 Intake air butterfly valve for the initial stage of start up period
PIC-1 Initial intake air suction pressure, then suction head pressure for flue gas recirculation
PIC-2 Suction pressure upstream of flue gas recirculation blower
QI-1 Gas analyzer for oxygen, carbon dioxide and residual nitrogen
PV-2 Purge butterfly gas valve over to the chimney
PV-3 Flue gas recirculation butterfly valve in charge for flue gas recirculation downstream of the blower post the over-ride control loop in effect
PV-4 Discharge of CO₂-rich CO2-Stream out of the flue gas recirculation loop to CO2-CC section
QIC-1 Oxygen analyzer in the flue gas oxy-fueling line
QV-1 Oxygen supply valve from the HPLTE-SG
PV-2 Discharge valve of the oxygen-rich off gas downstream of the CO2-CC section

TABLE 2
Typical flue gas composition of the flue gas of a conventional 500 MW coal power plant
with rough balance post CPCE integration with recirculation of flue gas and addition of
oxygen from HPLTE-SG, referred to Flue gas oxy-fueling

| | Streams as in FIG. #11 | | | | | | | |
|---|---|---|---|---|---|---|---|---|
| | (14) | | 13 | | 17 | | | |
| | Conventional coal power | | Flue gas | | CO₂-enriched | | | |
| | plant flue gas | | downstream | | gas CO2-Stream to | | 16 | |
| | (at start up of the plant) | | of CPCE's scrubber | | CO2—CC | | Recirculated flue gas | |
| Flue gas constituent | Nm³/h | Vol % | Nm³/h | Vol % | Nm³/h | Vol % | Nm³/h | Vol % |
| Nitrogen | 797112 | 67% | 0 | 0% | 0 | 0% | 0 | 0% |
| Oxygen | 59708 | 5% | 59708 | 8.0% | 25819 | 8% | 66480 | 8% |
| Carbon dioxide | 294336 | 25% | 1091448 | 91.8% | 296396 | 91.8% | 763175 | 92% |
| Water moisture | 42282 | 4% | 1234 | 0.107% | 345 | 0.11% | 889 | 0.11% |
| Inerts as Argon | 4926 | 0.41% | 1226 | 0.05% | 161 | 0.05% | 416 | 0.05% |
| Carbon monoxide | 0 | 0 | 123 | 0.01% | 16 | 0.01% | 42 | 0.01% |
| Gas stream | 1198364 | | 1153739 | | 322738 | | 831001 | |
| | | | | | 27% | | | |

| Streams as in FIG. #11 | |
|---|---|
| | 19 |

| | | | Oxygen added | | Oxygen added | | Oxygen-rich vent | |
|---|---|---|---|---|---|---|---|---|
| | | | recirculated flue gas | | recirculated to flue | | gas downstream of | |
| | Excess oxygen from | | u/s of CO-Oxidation | | gas d/s of CO- | | CO2—CC to CO₂ | |
| | HPLTE-SG for export | | reactor | | Oxidation reactor | | absorber | |
| Flue gas constituent | Nm³/h | Vol % | Nm³/h | Vol % | Nm³/h | Vol % | Nm³/h | Vol % |
| Nitrogen | 294336 | 100% | 0 | 0% | 0 | 0% | 0 | 0% |
| Oxygen | | | 257208 | 25.2% | 257187 | 25% | 25819 | 92% |
| Carbon dioxide | | | 763175 | 74.7% | 763175 | 75% | 2075 | 7% |
| Water moisture | | | 889 | 0.087% | 889 | 0% | 0 | 0% |
| Interts | | | 416 | 0.041% | 416 | 0% | 161 | 1% |
| (N₂ of coal's origin) | | | | | | | | |
| Carbon monoxide | | | 42 | 0.004% | 0 | 0% | 16 | 0% |
| Gas stream | | | 1021729 | 100% | 1021667 | | 28071 | |
| | Streams as in FIG. #11 | | | | | | | |
| | | | | | | | 3 | |
| | Rest gas downstream of | | Dehydrated rest gas | | | | Re-gasified LOX to | |
| | the CO₂ vent gas | | upstream of | | GOX downstream of | | heat recovery & back to | |
| | absorber | | expansion for LOX | | the expansion | | flue gas oxy-fueling | |
| Flue gas constituent | Nm³/h | Vol % | Nm³/h | Vol % | Nm³/h | Vol % | Nm³/h | Vol % |
| Nitrogen | 0 | 0% | 0 | 0% | 0 | 0% | 0 | 0% |
| Oxygen | 25303 | 93% | 25303 | 99% | 2935 | 94% | 22368 | 100% |
| Carbon dioxide | 2 | 0.01% | 2 | 0% | 2 | 0% | 0 | |
| Water moisture | 1813 | 7% | 0 | 0% | 0 | 0% | 0 | |
| Interts | 161 | 1% | 161 | 1% | 161 | 5% | 0 | |
| (N₂ of coal's origin) | | | | | | | | |
| Carbon monoxide | 16 | 0% | 16 | 0% | 16 | 1% | 0 | |
| Gas stream | 27295 | | 25482 | | 3115 | | 22368 | |

### Embodiment (II): Specifics and Points of Reference in the Parent Patent's Post-Combustion CO2-Stream from the Single-Cycle and the Combined-Cycle Gas Turbine Plants

The detailed description of present process invention discloses the integration of CO2-Stream, pretreatment of the CO2-Stream, preparation of CO₂-lean flue gas to CO₂-rich flue gas from the gas turbine power generation plants, i.e. the gas turbine single cycle plants as well as gas turbine combined cycle power plants.

This detail disclosure references to the post-combustion carbon capture, which has been disclosed in part in the FIG. 2, equipment 2, 4, 5, 7 (in the first and second stages) with their respective elements, and also to the process streams 1, 3, 6, 40 (downstream of the first stage).

The post-combustion carbon capture in present patent in continuation references also to the FIG. 3 with the stream 58.

One of the pertinent block diagram for the description in embodiment (II) relates to the block diagram, presented in FIG. 8 with the boundary limit I for the post-combustion process section and the streams 1, 2, 3, 8, 9, 16, 17, 18, 25, and in part the stream 27.

The other pertinent block diagram for this present patent in continuation relates to the block diagram presented in FIG. 9 with the boundary limit I for the post-combustion process section and streams 1, 2, 3, 4, 5, 6, 13, 14, 15, 22, 23, and in part for stream 24.

The embodiment (II) comprises the following seven specific features of the Advanced Combustion and steps from the start up of the gas turbine power plant, then up to the normal mode of operation at 100% load in the processing, including the augmentation case and de-sooting procedure, i.e.:
1) The CO₂-lean flue gas recirculation of the gas turbine power plants in order to obtain a CO₂-rich CO2-Stream upstream of the subsequent CO2-CC section
2) Oxy-fueling of the recirculated flue gas, i.e. adding pure oxygen produced in the HPLTE-SG into the recirculated flue gas in order to reduce the nitrogen; rather than, eliminate the presence of nitrogen from the intake combustion air for the normal mode of operation totally
3) Integration of other parent patent's internal CO2-streams into the flue gas recirculation and flue gas oxy-fueling line
4) The proceeding for the integration of parent patent's flue gas oxy-fueling in the both types of the gas turbine power plants that includes the execution of the start up period
5) Reduction of intake gas flow subject to gas turbine's compressor of ca. 30% to 35% gas in an existing plant, which stems from the by-pass recirculation flue gas to the HRSG and to the other part, because of addition middle pressure oxygen-rich purge gases and the regassified oxygen directly to the ring distributor of the gas turbine
6) Integration of other CO2-Streams of minor gas flow rate in order to achieve the Zero-CO2-Emission Fossil Energy
7) The operation of two over-ride process control loops for the gradual transitional execution is employed in the embodiment (II) from the cold start, then up to the full load operation of the plant, which comprises also the intermittent de-sooting and augmentation stages

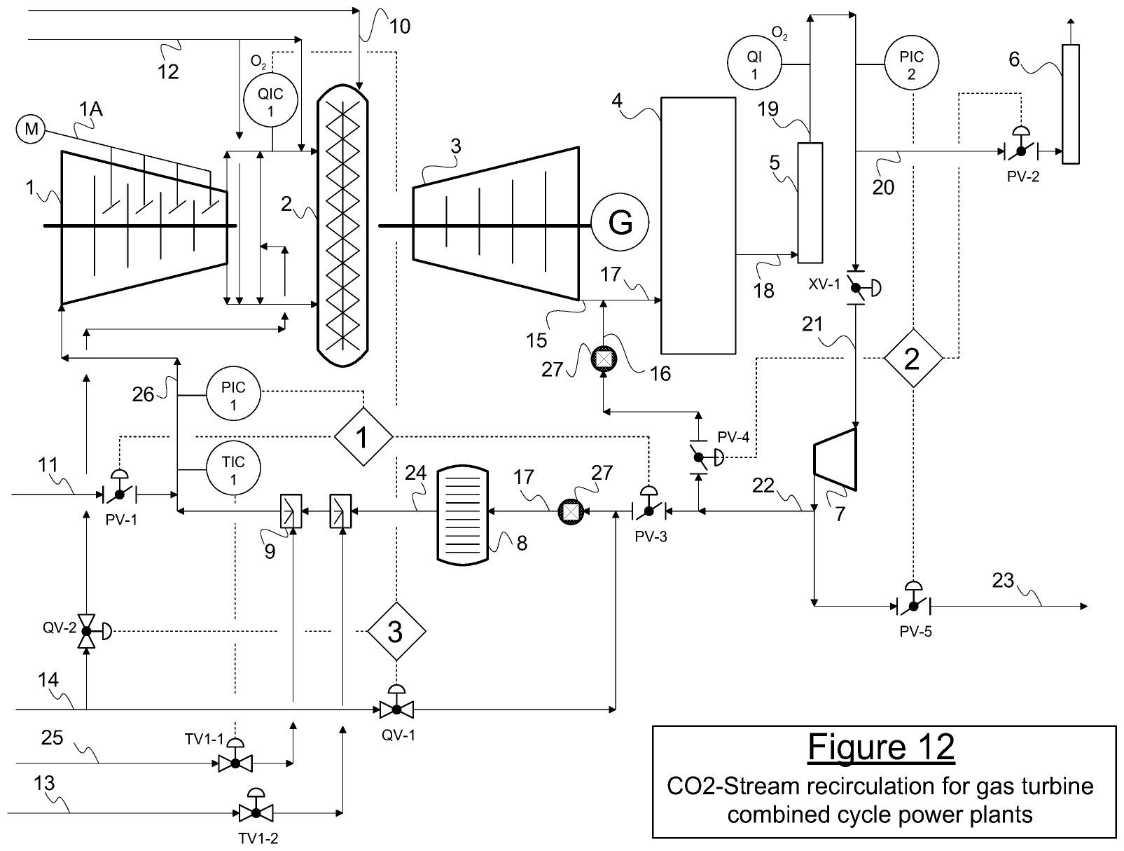

### BRIEF DESCRIPTION OF THE EMBODIMENT (II) VIA FIG. 12

The Advanced Combustion with flue gas recirculation and flue gas oxy-fueling for the CO₂ enrichment in post-combustion CO2-Streams in the flue gas of gas turbine power plants upstream of the CO2-CC section is presented by the brief description and FIG. 2 without any limitation to other embodiment(s).

The FIG. 12 shall present a typical gas turbine combined cycle power plant that operates conventionally with any fossil fuel gas, typically natural gas fed by the line 10 into the combustion compartment of the gas turbine, aka the ring distributor 2. The gas turbine plants currently on operation, run by intake combustion air 11 that supplies the combustion from the oxygen constituent of the air of about 21 Vol % (with almost remaining useless nitrogen in ca. 78 Vol %). Thus the typical flue gas downstream of HRSG in these plants provides a CO2-Stream with only about 4 Vol % to 6 Vol % CO₂ and 12 Vol % to 14 Vol % excess oxygen in dry flue gas.

For this purpose, the plant shall start first with the initial startup load of the natural gas 10 and the intake air 11 at the minimal load case with the adjustable blades 1A CLOSED as usual. The intake air 11 is compressed in 1, routed to the combustion chamber 2, wherein the natural gas 10 is added to and ignition takes place. The hot gas is routed then to the turbine compartment 3 that runs the generator. The hot flue gas 15 post turbine compartment is routed to the HRSG 4 in the gas turbine combined cycle plant, wherein steam is generated to run the steam turbine (not depicted in the FIG. 12) for generation of additional power.

After the present process invention implemented in the gas turbine combined cycle plant, the hot flue gas 18 post HRSG 4 will be routed first to the scrubber 5 (i.e. the equipment 5 in the FIG. 2). The cleaned de-sooted flue gas 19 is also almost dehydrated in the course of scrubbing up to a prevailing temperature of ca. 80 Degree of Fahrenheit at the cold end of scrubber. By then, shortly after the ignition is carried out, the CO2-Stream of the flue gas 19 with lean CO₂ concentration is rerouted by nearly OPEN XV-1 via stream 21 to the recirculation blower 7, while portion of the nitrogen containing flue gas 20 (observed by QI-1 in the CO2-Stream) is purged through the partially OPEN PV-2 to the stack 6.

The recirculated CO2-Stream 22 downstream of the blower 7 is first passed entirely through PV-3 OPEN position, while PV-4 stays still CLOSED. At the initial start up period, the entire oxygen 14 to the gas turbine at the initial minimal load with the CLOSED adjustable blades 1A, is supplied from the CO₂ liquid storage tank and the water storage tank over the electrolysis in the HPLTE-SG. These storage tanks supply the first units of HPLTE-SG that feeds back with the needed oxygen for flue gas oxy-fueling through the line 14 and QV-1 in the initial period of operation.

The recirculated flue gas 22 is added with the oxygen by line 14, which may be adjusted in temperature by a heat exchanger or more preferably by H₂/O₂-torch in normal mode of operation (fed with NG/O₂ during the start up period) 27 up the desired reaction temperature required in stream 17 for the CO-oxidation in 8. During the start up period the valve PV-4 is CLOSED, thus the line 16 does not by-pass portion of the recirculated flue gas to the HRSG.

Downstream of the CO-oxidation reactor the temperature of the recirculated flue gas 24 will be lowered to the degree compatible to the permissible intake temperature of the compressor 1 by injection of LOX (e.g. from the GOX expansion post the CO₂-absorber and dehydration).

Because the valve PV-4 is CLOSED for the by-pass flue gas 16 to the HRSG 4 during the ramp up period, the compressor 1 operates with the designated intake load gas flow 26 (i.e. streams of 11+24+25) in the start up period.

The permissible gas intake temperature is controlled by the LOX line 25 and the TV1-1 primarily, controlled by TIC-1 in the desuperheater 9. If the maximum permissible intake temperature can not be reached due the available balance of LOX, additional liquid CO₂ shall be injected additionally by TV1-2 via line 13 if needed for an existing gas turbine.

Typically, under the minimal load case of the gas turbine and the size of the HRSG, the nitrogen constituent of the flue gas should have declined to near zero as a result of gradually CLOSING intake air valve PV-1 for 11, and the purging the nitrogen via line 20 trough PV-2 in to the stack within a margin of time for two to four hours (observed with the gas analyzer QI-1).

Once the nitrogen constituent of the flue gas QI-1 declines to zero, the first over-ride control loop can be activated, wherein the PV-1 and PV-2 will be CLOSED gradually, XV-1 sets in full OPEN position, while the recirculation of the flue gas 16 into the HRSG can be commenced via OPENING of the PV-4 now. This side stream 16 is necessary in an existing plant, because the integration of the present process invention leads to about 30% to 35% less intake gas through the compressor 1. As result of this, portion of the recirculated CO2-Stream 16 must be routed to the HRSG after it is superheated by a supplementary firing with O₂/H₂ torch 27 in the normal mode of operation. In the commencing period, the torch(es) are fed with the natural gas and pure oxygen from the HPLTE-SG. By this way, the side stream 16 (typically about 30% of the recirculated flue gas) can be adjusted with the hot gas temperature from the gas turbine 15 in order to balance out the required gas flux 17 impinged to the existing HRSG 4 in the margin of the temperature as before the integration of Advanced Combustion. Once the plant stabilized, the torch(es) 27 can be fed with H₂/O₂ gases from the HPLTE-SG.

In a new plant, there is no side stream 16 necessary due the compact design of the plant for the same gross output. The gas intake flow 26 to the compressor 1 for the new plants is typically reduced to about 30% that serves to more power shift to the turbine 3, thus it delivers more electricity from the generator.

Traditionally, depending on the size of the HRSG, at this window of time, there is 80 Vol % to 90 Vol % CO₂ with the residual oxygen in the margin of 20 Vol % to 10 Vol % in the stream 19, respectively also in the streams 21, and 22. At this juncture, the second over-ride control loop can be activated, whereas the control valve PV-5 OPENS the CO₂-rich CO2-Stream 23 to the CO2-Stream's compressor in the CO2-CC section. By then, the further gradual increase of load can be continued until the full load of the gas turbine and the steam turbine of the HRSG.

Under increasing load of the gas turbine, other oxygen-rich process intern streams 12 like that from the CO2-CC section (i.e. streams 43 and 44 of the FIG. 2) can be routed to the ring distributor 2. By this way, a further relief of the compressor section 1 can be achieved that results in more power output through the generator. Similarly, once the full load mode has reached, the main oxygen supply 14, now consisting of 14(A) and 14(B) for flue gas oxy-fueling can be directed through the valve QV-2 to the ring distributor that is controlled by QIC-1 in the circuit 3 for the QV-1 and QV-2.

The non-condensable gases (like from the traces of nitrogen that would concentrate in the flue gas recirculation) will be purged downstream of CO2-CC section from the oxygen-rich gas, CO₂-absorber, GOX expansion, and regain of LOX. The inert components will be discharged to the atmosphere downstream of the latter expansion along with remaining GOX (these sections are not depicted in the FIG. 12).
FIG. 12 CO2-Stream recirculation for gas turbine combined cycle power plants

### ELEMENTS IN THE FIG. 12

| | |
|---|---|
| 1 | Compressor compartment of the gas turbine |
| 1A | Adjustable blades of the compressor |
| 2 | Ring distributor of the gas turbine (figuratively streched out in |
| | the arrangment to present the side streams) |
| 3 | Expander turbine compartment of the gas turbine |
| 4 | HRSG, Heat Recovery Steam Generation section |
| 5 | Scrubber of the present process invention (i.e. equipment 2 and |
| | 5 in the FIG. 2) |
| 6 | Stack |
| 7 | Flue gas recirculation blower |
| 8 | Catalytic carbon monoxide oxidation reactor for oxidation of |
| | CO traces |
| 9 | Liquid CO₂ injection for recirculated flue gas direct cooling, |
| | optionally with a heat exchanger in place |
| 10 | Fossil fuel gas, i.e. natural gas, process combustion gas, syngas, |
| | optionally enriched with hydrogen |
| 11 | Intake air for the initial start up period of the gas turbine for the |
| | initial minimum load |
| 12 | Regasified LOX downstream of oxygen-rich expansion of the |
| | off gas from the CO2-CC section (equals to the stream 44 in |
| | FIG. 2), routed into the ring distributor of the gas turbine |
| 13 | Liquid CO₂ desuperheating line for temperature control |
| 14 | Injection of primary oxygen 14 (A) and secondary oxygen 14 |
| | (B) as pure oxygen from the HPLTE-SG into the recirculated |
| | flue gas in the normal mode of operation |
| 15 | Hot flue gas downstream of the gas turbine and upstream of the |
| | HRSG |
| 16 | Recirculated hot flue gas for compensation of the hot flue gas |
| | flux to the HRSG upstream of reheating torch(es). The hot flue |
| | gas from the gas turbine stream 15 and the reheated recirculated |
| | flue gas stream 16 combined, shall be tantamount in volumetric |
| | flux to the HRSG at a rate equal to the flux prior to the |
| | integration of parent patent in an existing plant |
| 17 | Oxy-fuled recirculated flue gas, reheated to meet the required |
| | reaction temperature for the carbon monoxide oxidation, |
| | upstream of CO-Oxidation reactor; termed flue gas conditioning |
| 18 | Flue gas to the parent patent's scrubber, e.g. two stage scrubber |
| | (depicted as equipment 5 in FIG. 2) |
| 19 | Cleaned flue gas at the cold end of the scrubber |
| 20 | Purge flue gas to the stack during the start up period |
| 21 | Recirculated CO2-Stream upstream to the flue gas recirculation |
| | blower |
| 22 | Recirculated CO2-Stream downstream of the flue gas |
| | recirculation blower |
| 23 | CO₂-rich CO2-Stream upstream to compression for the |
| | CO2-CC section |
| 24 | Recirculated oxygen-enriched, CO-free flue gas upstream of |
| | desuperheater(s) for temperature controlled cooled CO2-Stream |
| | upstream of intake suction head of the gas turbine's |
| | compression compartment 1 |
| 25 | LOX, liquid O₂ injection for desuperheating for the temperature |
| | adjustment upstream of intake suction head of the gas turbine's |
| | compression compartment 1 |
| 26 | Gas analyzer for residual oxygen, water vapor by dew point |
| | analyzer, and for the nitrogen at the initial start up period |
| 27 | Supplementary firing for reheating of the recirculated flue gas |

### FURTHER ELEMENTS IN FIG. 12

14 (A, B) The oxygen streams 14 (B) balances out the need for the additional oxygen (the stochiometric ½ O₂), needed in the flue gas oxy-fueling of the natural gas fired power plants, i.e. for the gas turbine, CHP and the natural gas fired boilers.
   The stream 14 shall present the primary oxygen 14 (A) that is obtained from the primary carbon dioxide in the HPLTE-SG that stems from the carbon of the fuel, i.e. natural gas in the gas turbine. The stream 14 (B) refers to the secondary oxygen that is obtained from the secondary carbon dioxide, which is regained from the water shift convertion of the syngas after the carbon dioxide separation from the hydrogen in the CO2-CC section.
   Further detail and interlinked water shift converters and streams in the FIG. 3 are presented in this patent in continuation in the section for abbreviations, special technical terms and expressions below.
PIC-1 Intake suction pressure head of the gas turbine compression compartment, employed for the first over-ride control loop with the control valve PV-1 for the initial intake air and the PV-3 control valve for CO2-Stream recirculation. PIC-1, PV-1, and PV-3 with the adjustable blades 1A are in charge for the first over-ride control loop.
QIC-1 Oxygen analyzer to maintain the control loop for QV-1, preferably in the initial start-up period, by then for QV-2 for injection of sufficient oxygen into the ring distributor and combustion chamber of the gas turbine directly
PIC-2 Pressure control measurement for the over pressure suction head of the CO2-Stream recirculation blower 7, and the operation of the purge gas control valve PV-2 in the start up period. PIC-2 directs the second over-ride control loop in the normal mode of operation for control valves PV-4 and PV-5.
XV-1 Remote control valve, partially in OPEN in MANUAL position during the start up period of the plant, by then fully OPEN in the normal mode of operation

TABLE 3
Typical flue gas composition of gas turbine combined cycle power plant with rought balance
post CPCE integration with recirculation of flue gas and addition of
oxygen from HPLTE-SG, referred to Flue gas Oxy-Fueling

| | | | | | 22 | | 23 | | 16 | |
|---|---|---|---|---|---|---|---|---|---|---|
| | Typical flue gas | | 21 | | Recirculated flue gas to | | CO₂-enriched flue | | Recirculated flue gas | |
| | exhaust gas of | | Flue gas d/s of | | oxy-fueling conditioning | | gas CO2-Stream to | | routed to the HRSG in | |
| | GTCC | | CPCE's scrubber | | and reheating | | CO2—CC | | addition to stream #15 | |
| Flue gas | Nm³/h | Vol % | Nm³/h | Vol % | Nm³/h | Vol % | Nm³/h | Vol % | Nm³/h | Vol % |
| CO₂ | 50400 | 4.6% | 852118 | 79% | 794548 | 79% | 50400 | 79% | 238365 | 0.79 |
| O₂ | 124875 | 11% | 124875 | 12% | 117426 | 12% | 7449 | 12% | 35228 | 12% |
| H₂0 | 110880 | 10% | 81392 | 0.05% | 503 | 0% | 32 | 0.05% | 151 | 0% |
| N₂ | 801321 | 73% | 0 | 0% | 0 | 0% | 0 | 0% | 0 | 0.00% |
| CO | 12 | 0.001% | 12 | 0.001% | 11 | 0.001% | 1 | 0% | 3 | 0.00% |
| Inerts | 11157 | 1.02% | 11157 | 0.87% | 8750 | 0.87% | 555 | 0.87% | 2625 | 0.0087 |
| Gas stream | 1098645 | | 1069555 | | 1005758 | | 63797 | | 301727 | |
| | | | | | | | 6% | | 30% | |
| | | | | | 17 | | 24 | | 26 | |
| | Primary Oxygen | | Add-to O₂ from the | | Recirculated | | Oxygen added | | Recirculated oxy-flue | |
| | from HPLTE-SG | | Secondary Oxygen | | oxygenized | | recirculated flue | | gas conditioned with | |
| | for flue gas | | from HPLTE-SG for | | preheated flue gas to | | gas d/s of CO- | | regasified LOX d/s | |
| | oxy-fueling | | flue gas oxy-fueling | | CO Oxidizer | | Oxidation reactor | | CO Oxidizer | |
| Flue gas | Nm³/h | Vol % | Nm³/h | Vol % | Nm³/h | Vol % | Nm³/h | Vol % | Nm³/h | Vol % |
| CO₂ | | | | | 556184 | 71% | 556376 | 71% | 556376 | 71% |
| O₂ | 75600 | 100% | 26244 | 100% | 218600 | 28% | 218596 | 28% | 222596 | 29% |
| H₂0 | | | | | 320 | 0.04% | 320 | 0.04% | 320 | 0.04% |
| N₂ | | | | | 0 | 0% | 0 | 0% | 0 | 0% |
| CO | | | | | 7 | 0% | 0 | 0% | 0 | 0% |
| Inerts | | | | | 5570 | 0.71% | 5570 | 0.71% | 5570 | 0.71% |
| Gas stream | | | | | 780681 | | 780863 | | 784863 | |
| | Oxygen-rich vent | | Rest gas downstream | | Dehydrated rest gas | | | | Re-gasified LOX | |
| | gas downstream of | | of the CO₂ vent | | upstream of | | GOX downstream of | | back to flue gas | |
| | CO2—CC | | gas absorber | | expansion for LOX | | the expansion | | oxy-fueling | |
| Flue gas | Nm³/h | Vol % | Nm³/h | Vol % | Nm³/h | Vol % | Nm³/h | Vol % | Nm³/h | Vol % |
| CO₂ | 397 | 4.7% | 2 | 0.01% | 2 | 0.02% | 2 | 0% | | |
| O₂ | 7449 | 89% | 7449 | 87% | 7449 | 93.04% | 670 | 55% | 6778 | 100% |
| H₂0 | 0 | 0 | 574 | 7% | 0 | 0.00% | 0% | 0% | | |
| N₂ | 0 | 0 | 0 | 0% | 0 | 0.00% | 0% | 0% | | |
| Inerts | 555 | 7% | 555 | 6% | 555 | 6.93% | 555 | 45% | | |
| Gas stream | 8401 | | 8580 | | 8006 | | 1227 | | 6778 | |

### Embodiment (III): Specifics and Points of Reference in the Parent Patent's Pre-Combustion Lean CO2-Stream from the HPLTE-SG and Gasification

The embodiment (III) of present patent in continuation presents the specific features of the CO2-Stream in pre-combustion carbon dioxide capture to obtain liquid carbon dioxide from the syngas generated from the gasification process, yet more specifically from the cathodic syngas of the HPLTE-SG of the parent patent, first in composition of CO/2H₂, and then in CO₂/3H₂ composition downstream of the water shift converter(s).

The embodiment (III) includes also the pre-combustion carbon capture from the secondary CO2-Stream that stems from the carbon of the cathodic CO/2H₂ stream downstream of the HPLTE-SG, and then through the HP or IP water shift converters. Thus the secondary CO2-Stream consists in CO2/3H₂ composition (as disclosed in FIG. 3 form the HP water shift convereter in the Stream 37; likewise disclosed in FIG. 3 from the IP water shift convereter in the stream 48).

This lean secondary CO2-Stream in turn, originates either downstream of the HPLTE-SG through the HP water shift converter (i.e. stream 37 in the FIG. 3 downstream of HP water shift converter 35), or it originates from the IP water shift converter (i.e. stream 48 in FIG. 3 downstream of the IP water shift converter 46). Either CO2-Streams CO₂/3H₂ is routed to HP or IP CO2-CC, whence HP secondary liquid CO₂ is obtained (FIG. 3, stream 38) or secondary IP liquid CO₂ is gained (FIG. 3, stream 49).

The points in reference to the CO2-Stream in the embodiment (III) for pre-combustion carbon capture from the gasification process or HPLTE-SG are interconnected with:
a) FIG. 1 for the CO2-Stream from the high-pressure gasification, streams 1, 2, 3, 4, 5, 6, 7, and 17, whereas the present disclosure pertains to the details taking place along the stream 7 in FIG. 1
b) FIG. 7 for the CO2-Stream from the high-pressure gasification is indicated in streams 1, 2, 3, 4, 5, more specifically 6. The present detail disclosure presents the specific features taking place along the line 6 in the block diagram FIG. 7
c) FIG. 6 for CO2-Stream from the middle-pressure and low-pressure gasification is indicated in streams 1, 2, 3, 4, 5, 6, and particularily in the block for 7. The present disclosure presents the specific features taking place along the lines 6 and the block 7 in the block diagram FIG. 6
d) The FIG. 10 in the Boundary Line I, stream 1, whereas the present detail disclosure the specific features taking place along the line 1.

The present patent in continuation implies the peculiarities of the present process invention with regard to CO2-Stream treatment from the syngas by removal of CO₂ from the syngas that considers the proceeding with the co-absorbed carbon monoxide, fine purification of CO2-Stream containing CO₂ and carbon monoxide from the undesired traces of water and selexol in that stream, which takes place in the scavenger adsorber column, then the separation of carbon dioxide by liquefaction of carbon dioxide from CO₂/CO/H₂ gas stream in the parent patent's CO2-CC section, by then the rerouting of the offgas from the CO2-CC section consisting primarily of carbon monoxide with minor portion of carbon dioxide back to the syngas proceeding. These peculiarities are summarized as follows:
1) The obtained syngas post the gasification's sections for syngas cleaning, syngas cooling and syngas dehydration, or from HPLTE-SG i.e. CO₂/CO/H₂ gas stream is passed through a CO₂-absorbing solvent in a preferably in two-stage physisorption Acid Gas Removal that is the selexol or rectisol solvent, Benfield or any other CO₂ separation for the treatment of the CO2-Stream
2) The CO₂-laden solvent with little portion of co-absorbed CO in the selexol or rectisol undergoes flash desorption and thermal desorption, wherein the CO₂-rich CO2-Stream is obtained with little portion of CO
3) The CO₂-rich CO2-Stream with little portion of CO is then purified from the water and selexol traces
4) The CO₂-rich CO2-Stream with portion of CO is passed to the parent patent's CO2-CC for cooling and separation of CO₂, preferably at supercritical pressure by condensation, whereas the separated liquid carbon dioxide from the CO2-CC is further processed to the parent patent's HPLTE-SG section
5) The offgas downstream of the CO2-CC section with the non-condensing CO and residual of CO₂ is then recompressed and returned back to the main syngas stream for further processing, like for production of high-value hydrocarbons

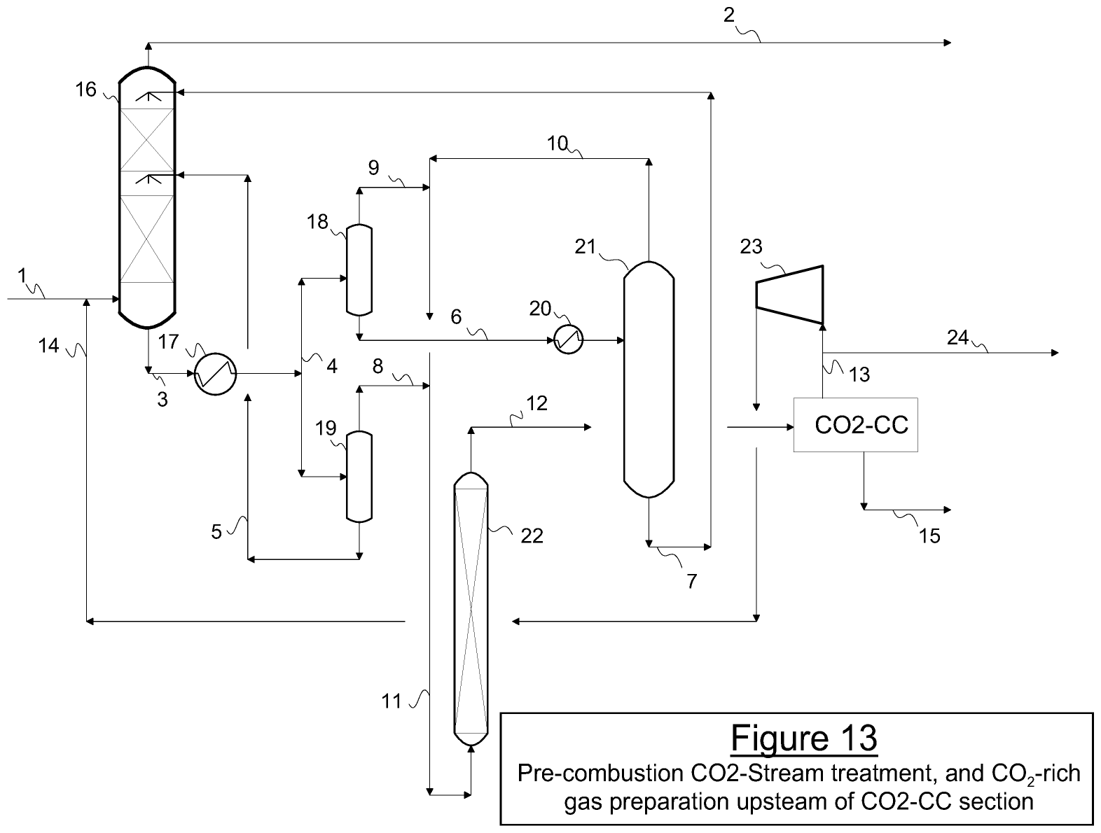

### BRIEF DESCRIPTION OF THE EMBODIMENT (III) VIA FIG. 13

The specifics of the embodiment (III) is described more extensively via the FIG. 13. The present patent encompasses for the pre-combustion CO2-Stream 1 from the gasification and/or from the HPLTE-SG's cathodic syngas CO/2H2 and/or any CO2-Stream(s) that is obtained from the redeployed secondary liquid carbon dioxide (like presented in FIG. 3, streams 38 and 49), as well as any CO2-Stream post water shift converters in CO2/3H2 composition.

The CO2-Stream 1 can be compressed (it equals the element 7 in FIG. 6) to a higher pressure, in case the syngas stems from the MP-, or LP-gasification process. If the CO2-Stream 1 is produced via high pressure gasification, it can be processed for further treatment directly without compression. The treatment of the CO2-Stream according to this patent in continuation includes CO₂ absorption process that is carried out preferably in a two stage absorption column 16, whence the CO₂-free syngas 2, mainly consisting of H₂/CO can be processed to further processing.

If the syngas stems from HPLTE-SG with the following water shift converters, then the stream 7 consists of hydrogen alone for chemicals like NH₃ or as it is considered for the super-efficient hydrogen based fossil power generation via Second Bairamijamal Cycle (FIG. 10).

The absorbed carbon dioxide of the CO2-Stream 3 with the co-absorbed carbon monoxide in the selexol can be preferably preheated through the heat exchanger 17 before splitting in two streams 4, one for selexol rough regeneration by use of flash desorption in the flash drum 19 only. The other side dstream from 4 is processed to regeneration via (i) flash desorption 18, and then the (ii) thermal desorption 6. The fine regeneration of the selexol 6 from the flash desorption 18 is then preheated through the heat exchanger 20, by then regenerated in the thermal desorption column 21. The desorption gases, i.e CO₂-rich/containing CO in lesser portion from the flash drums 18 and 19, are converge with the CO₂-rich/CO containing desorption gas 10 from the thermal desorption column 21 are all routed together in the stream 11 for the fine purification of the CO₂-rich/CO-containing gas through the scavenger adsorption column 22.

The treated CO₂-enriched purified CO2-Stream 12 can be routed now to the parent patent's CO2-CC section, wherein the CO₂ is separated from the CO₂/CO-gas stream 12 by way of liquefaction, whereas the liquid carbon dioxide 15 can be directed to the parent patent's HPLTE-SG section. The remaining CO-rich off gas 13 can be recompressed with 23 and rerouted back to the syngas proceeding through the stream 14. The none-condensing gases, particularly in the start up period, is vented by the stream 24.
FIG. 13 Pre-combustion CO2-Stream treatment, and CO₂-rich gas preparation upstream of CO2-CC section

### ELEMENTS IN THE FIG. 13

| | |
|---|---|
| 1 | CO2-Stream from the syngas CO/2H₂ i.e. from the HPLTE-SG |
| | post water shift in mainly CO₂/3H₂ composition, and/or from HP-, |
| | MP, and LP gasification, downstream of syngas compression, gas |
| | clean up section, water-shift converter(s) |
| 2 | Cleaned CO₂-free hydrogen for downstream production plants e.g. |
| | NH₃ and for the new generation of power plants super-efficient |
| | hydrogen based fossil power plants (vide Second Bairamijamal |
| | Cycle) |
| 3 | Selexol or rectisol or other chemical agents for Acid Gas Removal, |
| | absorbed with CO₂ and to lesser degree co-absorbed CO |
| 4 | Preferably preheated, splitted streams for regeneration of absorbens |
| 5 | Partially CO₂-free and CO-free selexol downstream of the flash |
| | vessel 19 back to lower compartment of absorber 16 |
| 6 | Flashed selexol upstream of the preheater 20 and the CO₂/CO |
| | stripper column 21 |
| 7 | CO₂-free and CO-free selexol regenerated, cooled down and |
| | rerouted back to the fine-absorption section of the absorber column |
| | 16 |
| 8 | CO₂-rich CO2-Stream with little portion of co-absorbed CO from |
| | the flash drum 19 |
| 9 | CO₂-rich CO2-Stream with little portion of co-absorbed CO from |
| | the flash drum 18 |
| 10 | CO₂-rich CO2-Stream with little portion of co-absorbed CO from |
| | the desorber column 21 |
| 11 | Collected CO2-Streams with residual traces of water and selexol |
| | upstream of water-, selexol to the scavenger adsorber 22 |
| 12 | Cleaned CO2-Stream free of traces of water and selexol to the |
| | CO2-CC section with little portion of carbon monoxide |
| 13 | CO-rich offgas with little portion of CO₂ from the CO2-CC section |
| | to the interim recompressor 23 |
| 14 | CO-rich offgas with little portion of CO₂ back to the processing |
| | upstream to the column 16, in case syngas is desired for the |
| | hydrocarbon productions |
| 15 | Liquid carbon dioxide from the pre-combustion CO2-Stream to the |
| | HPLTE-SG section |
| 16 | CO₂ absorption column, preferably selexol, rectisol or any other |
| | CO₂ absorber column |
| 17 | Preheater upstream of flash drum |
| 18 | Flash drum for fine regeneration of selexol |
| 19 | Flash drum for selexol for the rough separtion of CO₂/CO |
| 20 | Preheater upstream of fine-regeneration for selexol |
| 21 | Selexol regeneration column |
| 22 | Two multi-stage selexol-water scavenger adsorbers for removal of |
| | water and selexol traces up to ppb-level from the CO₂-rich |
| | CO2-Stream with little portion of carbon monoxide, operating |
| | intermittently. |
| 23 | Recompressor for CO-rich offgas with little portion of CO₂ |
| 24 | Purge gas to vent out the non condensing CO-rich gas if pure |
| | hydrogen desired |

### Embodiment (IV): Utilization of CO₂ Containing Streams for Additional Process Heat Generation for DC Back Up Power to HPLTE-SG and Additional Syngas Co-Production

The present embodiment presents a way to the utilization of the CO₂-enriched flue gas to generate process heat by use of gasification reactions for the CO2-HR and CO2-PG sections. The parent patent's disclosure references the process heat generated by use of chemical gasification reactions that had been presented for superheating and/or supraheating of carbon dioxide to high temperature in the new First Thermodynamic Cycle as part of the CO2-HR section. That process heat positions the recovery of available thermal energy or generating of process heat, which eventually is to generate primarily AC power by use of supercritical CO₂ turbine(s) in the CO2-PG section. With reference to the parent patent, that AC power was designated to either be dispatched to the grid or be converted to DC current for backing up the electrolysis in the HPLTE-SG section.

However, there were no further specific details disclosed in the parent patent regarding the kind of those process heat sources nor as regards to the gasification, which chemical gasification reactions were to be employed for process heat generation. The present embodiment discloses the kind of those process heat generations as process heat generated downstream of a gas turbine, i.e. the heat recovery section of a gas turbine (i.e. specifically a gas turbine combined cycle as an independent power island aside the boundary line of this process invention or specifically an auxiliary gas turbine combined cycle within the boundary of the present process patent) as well as the process heat generated by the chemical gasification reactions. The process heat generation by either of the three v.s. sources comprises in the present patent in continuation also the superheating, and/or re-superheating of the saturated steam gained from the adjacent syngas utilizing plant into the present high temperature process heat generation.

In this embodiment, a part of the CO₂-enriched recirculated flue gas is harnessed to generate high temperature process heat by way of the single-stage gasification reaction (7) or by way of two-stage gasification with (7) and (8) combined.

2CH₄+O₂+CO₂→3CO+3H₂+H₂O (7)

CH₄+H₂O CO+3H₂ (8)

In actuality, the overall reaction (7) is consisting of two parallel gasification reactions. One is the endothermic reaction of methane and carbon dioxide to form syngas in the composition [2CO/2H₂], also referred to as methane reforming, while the other is the exothermic gasification reaction of methane and oxygen to form [CO/H₂/H₂O].

The reaction (8) is the typical endothermic steam reforming reaction that is carried out by catalytic reaction under lesser mol stream of natural gas relatively to the mol steam stream of the steam in order to prevent the cocks formation on the catalyst's surface. Thus, the molar ratio of steam to methane is kept between 1.5 to 3.0 typically. In utmost all present-day syngas and hydrogen generating plants the two above reactions, i.e. the endothermic steam reforming, and then the partial oxidation reaction of methane with oxygen are carried out in two distinct reactors, i.e. the Primary Reformer and Secondary Reformer, respectively. If these two reactions were carried out in one reactor, yet in two compartments, then the reactor is referred as autothermal reformer.

The present embodiment employs the overall reaction (7) in a single-stage gasification reactor in order to attain high temperature process heat, typically between 850 Deg C. to 950 Deg C. that is then utilized to superheat, rather supraheat the carbon dioxide to high temperature, yet preventing the thermal decomposition of carbon dioxide at supraheated region, typically at higher than 1000 Deg C. Equally, the present embodiment uses the same high temperature process heat to generate superheated steam, preferably the saturated steam from the adjacent plant, wherein the generated syngas streams of the present process is fed to produce final products like methanol, ethanol, gasoline, aviation fuel, ammonia and other final products.

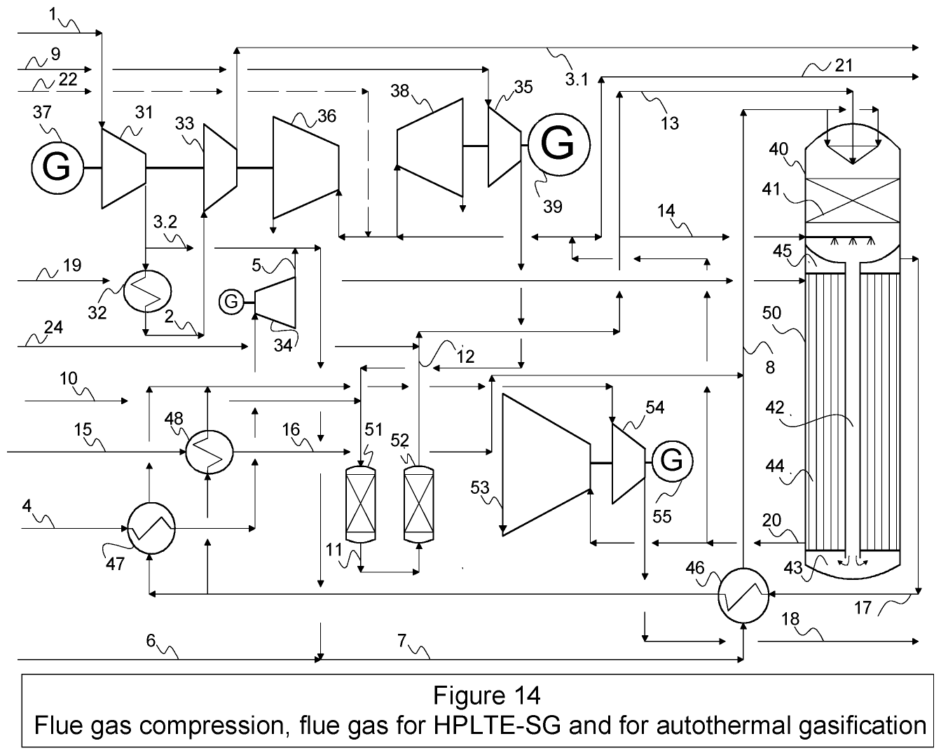

The catalytic reaction (8) is carried out in the present embodiment via a two-stage gasification reactor in a case to case option only. If the reaction (8) is to be considered (for instance to adjust the CO/H₂ ratio of the export syngas to the adjacent plant), then this embodiment considers the reaction (8) in a way that the molar ratio of the produced water through the reaction (7) stands to the natural gas reaching in to the chamber for the catalytic reaction (FIG. 14, element 42, then 43 into the catalytic tubes 44), i.e.

$$\frac{\dot{n} ( {H}_{2} O )}{\dot{n} ( {CH}_{4} )} = 1 .75 \text{∼} 2.8$$

more specifically:

$$\frac{\dot{n} ( {H}_{2} O )}{\dot{n} ( {CH}_{4} )} = 2.3 \text{∼} 2.5$$

Wherein the differential n presents the mol stream of each component at different load of the plant in steady state mode of operation.

The gasification of portion of the CO₂-enriched recirculated flue gas, particularly with other CO₂-containing gaseous process media, opens a new inventive advantageous way, distinguished with the following poignant reasons:
1. Available CO2-Stream at high concentration of the CO₂ by the present Advanced Combustion
2. CO2-Stream with the concomitant oxygen that is one of the reactants in all the gasification reactions
3. Integration of oxygen-rich CO2-Sidestream with typically 92 Vol % O₂, 7.5 Vol % CO₂ and 0.5 Vol % intert gaseous components (mostly carbon monoxide and nitrogen from the nitrogen constituent of the coal) in an advantageous way in terms of number of the equipment and volume of the investment, as well as operation costs
4. Integration of the Regasified-CO2 that has been deployed in the ACU units as condensing media from liquid CO₂ at nearly 75 bar and 31 Deg C. to ca. 45 bar at 10 Deg C. The present embodiment employs the Regasified-CO2 to gasification that operates at nearly the same pressure level, yet it can be harnessed for heat recovery, first for the CO₂ cooling upstream of the CO₂ main condenser, then preheated in the steam condenser of the steam-water cycle, by then, it recovers other process heat before it is fed to the gasification
5. Harnessing the available low costs IP oxygen from the HPLTE-SG that supplants the oxygen preparation from an air separation unit for the gasification
6. Integration of cold high pressure CO2-Sidestream, i.e. the purge gas downstream of the CO2-CC section at typically 9 Vol % to 10 Vol % of the CO₂-enriched flue gas from the Advanced Combustion and at ca. 32 Deg C. and a pressure of ca. 75 bar, which is harnessed for waste heat and process heat recovery, superheated, and then it drives the expander turbine down to a pressure at ca. 45 bar upstream of the gasification that enables to run generator for primary AC current for dispatch to the grid or post AC/DC conversion to back up the HPLTE-SG power supply
7. Yet, most importantly, the gasification of those CO2-Sidestreams above provides high temperature process heat for reheating, and superheating of CO₂ streams in the new First Thermodynamic Cycle, typically to 650 Deg C. and higher upstream of the supercritical CO₂ turbine(s),
8. Similarly, the high temperature process heat gained from the gasification is harnessed to superheat the saturated steam (typically from other parts of the plant, most importantly obtained from the adjacent plant downstream of syngas streams, i.e. methanol, ethanol, DME, aviation fuel, gasoline, other hydrocarbons, or ammonia production) for driving the steam turbine(s)

The above turbines are utilized to drive compressors (i.e. compressor for the recirculated flue gas, natural gas and others) as well as to drive generators for the primary AC current that can be either dispatched to the grid, or rather converted to DC current for back-up supply line for the HPLTE-SG electrolysis.
9. In addition to the above points, the gasification of CO2-Sidestreams generates a syngas stream out the single stage gasification process in ratio of 3CO-to-3H₂, or a syngas at higher hydrogen constituent, if two-stage gasification carried out.

The present embodiment references in the mass and energy balances to a carbon ratio that refers to the carbon mol stream of CO₂, which is deployed to the above gasification reactor to the carbon mol stream of the entire CO₂ from the Advanced Combustion. This embodiment employs a carbon ratio in the sense of this definition in the margin of 15% to nearly 25% for gasification typically.

Higher ratio of 25% for the gasification can be economically reasonable, if additional oxygen is available at a site, like if the generated syngas from the HPLTE-SG and/or from the gasification of CO₂-enriched CO2-Stream is be used for the ammonia production. Since in this case an air separation unit is needed to supply the nitrogen from the air, the off spin of that air separation unit can provide additional oxygen that then might reason the higher CO₂-enriched CO2-Stream to the gasification.

### BRIEF DESCRIPTION OF THE EMBODIMENT (IV) VIA FIG. 14

The present embodiment is described exemplarily via FIG. 14 and the list of elements below, yet without to restrict nor restrain the deployment of the processing in any limitation.

The flue gas post flue gas oxyfueling and recirculation from the Advanced Combustion 1 according to the Table 1 is routed to the compression section of the CO2-CC section that consists of subcritical flue gas compressor 31, and supercritical compressor 33 with respect to the discharge pressure of the compressors to the critical pressure of the carbon dioxide. The flue gas is split downstream of the subcritical compressor 31, whereas a portion of the flue gas 3.2 is utilized for preheating and gasification, while the main stream 3.1 of the flue gas is cooled by the intercooler 32, before the cooled flue gas 2 is directed to the supercritical compression 33 and eventually routed to the CO2-CC section for cooling, dehydration, and separation of the carbon dioxide by way of condensation.

The CO2-Sidestream 4 from the purge gas stream downstream of the CO2-CC's condenser(s), typically positioning ca. 10% of the CO₂-enriched flue gas to the CO2-CC section 3.1 and consisting of ca. 7.5 Vol % CO₂; 92 Vol % O₂ with the rest of inert gases (mostly carbon monoxide and nitrogen from the nitrogen constituent of the coal) at a pressure of ca. 75 bar and temperature of ca. 31 Deg C. This CO2-Sidestream has been passed through the CO2-HR heat exchangers (e.g. vs. steam condenser, and process heat exchanger), before it is further superheated by syngas heat exchanger 47 and directed to the expander turbine 34 that generates AC power by the attached generator, before it is joined at a pressure of 45 bar and temperature of ca. 40 Deg C. via stream 5 to the stream 3.2 for the gasification.

The other CO2-Sidestream, the Regasified-CO2 6, originally used as condensing media in the flue gas condensation by partial release of liquid CO₂ (ca. 5% of the entire liquid CO₂) from ca. 75 bar and 31 Deg C. to ca. 45 bar and 10 Deg C. is preheated in the CO2-HR for waste heat recovery and joins at ca. 75 Deg C. to the other CO2-Streams 3.2 and 5 forming the stream 7. The stream 7 is preheated by syngas process heat recovery heat exchanger 46 up to ca. 450 Deg C. in single-stage gasification or up to nearly 375 Deg C. in two-stage gasification. Depending on load and temperature gradients, an additional syngas process heat exchanger 49 (not depicted in the FIG. 14) may be installed between the gasifier effluent stream 17 and heat exchanger 46 for additional generation of superheated CO₂ or superheated steam, if needed.

The flue gas compressors 31 and 33 are driven typically by turbine 36 either by supercritical CO₂ turbine 36.1 or by an attached steam turbine 36.2, which also runs the generator 37 for generation of AC power.

Traditionally, the natural gas 9 for the generation of additional process heat shall be compressed with 35 for the gasification. Like the flue gas compressors, the natural gas compressor 35 is driven by a turbine 38, i.e. either by supercritical CO₂ turbine 38.1 or by an attached steam turbine 38.2, which also drives the generator 39 for generation of additional AC power.

A side stream of hydrogen 10 is routed to the compressed natural gas, before the hydrogenated natural gas is directed to the hydration reactor 51 to convert the sulfur constituents of the natural gas to H₂S in the stream 11. The H₂S and residue of hydrogen in the natural gas is further directed to desulfurization reactor 52, wherein the H₂S is reacted with the ZnO pellets to ZnS and water. The desulfurized natural gas 12 is further divided to the natural gas main stream 13, and 14 in case the two-stage gasification is to be carried out, and also to a side stream 13.1 for the ignition of the gasifier during the start-up period (not depicted in the FIG. 14).

The additional oxygen for the gasification is performed from the IP extraction of the anodic oxygen 15 from the HPLTE-SG streams post the CO2-HR sections for the closing trajectory of the CO₂ First Thermodynamic Cycle and preheated by heat recovery vs. steam in the steam condenser, then further superheated vs syngas process heat exchanger 48 and preheated in 16 up to ca. 185 Deg C. before it is joined to the CO2-Stream 7, forming the stream 8 with ca. 380 Deg C. upstream of the gasifier 40. The oxygen side stream 16.1 for ignition of the gasifier during the ramp up period is not depicted in the FIG. 14.

The reactants to the gasification 40 consists of oxygen-added carbon dioxide stream 8 and the natural gas 13, which react to syngas by the reaction (7) generating a high temperature process heat with the standards reaction enthalpy of 75 kJ/mol. This process heat is eventually released in the high temperature section of the gasifier 41 cladded with refractory material and internal recirculation flux before it is routed downwards through the reactor's central pipe 42 (typically installed for the autothermal gasifier with the static mixer elements for homogeneous mixing of the secondary natural gas 14) in order to redistribute in the reactor lower chamber 43 upstream of the integrated heat recovery section 50 with the reactor's heat exchanger tubes 44. The gasifier 40 is distinguished by the reforming catalyst placed inside of the tubes 44 in case the autothermal gasification is to be carried out with the accompanying endothermic steam reforming reaction (8).

The generated hot syngas in the gasifier is recollected in the compartment 45 before passing out as stream 17. The hot syngas 17 may passes through an outside installed heat exchanger 49 (not depicted in the FIG. 14) that regulates the load cases and balances out the fluctuations in the high temperature process heat consuming equipment (e.g. 36, 38, 53 or other consumers down to the stream 21). Finally, the hot syngas is routed to 46, 47, and cooled down in 48 for adjacent downstream production plants via 18 for production of final products. The syngas may be recompressed by the syngas recompressor 54, if high pressure syngas is required i.e. for water shift conversion, CO₂ pre-combustion removal for ammonia production. In this case, the syngas recompressor can be driven either by supercritical CO₂ turbine 53.1 or by an attached steam turbine 53.2, driving the generator 55 too.

The core objectives of high temperature process heat generation through the gasification of carbon dioxide and methane with the oxygen, primarily from the HPLTE-SG, comprises in this embodiment the 50 integrated in the reactor, with the optionally 49 installed outside of the reactor, which are employed to reheat the available supercritical superheated carbon dioxide (for instance at 175 bar, downstream of regenerative heat exchangers, and/or the saturated steam from the adjacent plant (e.g. saturated steam from methanol plant, typically at 47 bar with at least 260 Deg C, at massive mass throughput) up to a temperature field of 650 to 850 Deg C upstream of each turbine.

These re-superheating measures can be carried out by gasification and/or in combination with the heat recovery section of an auxiliary gas turbine, and/or further backed-up with supplementary firing via natural gas and oxygen and/or H₂/O₂ torches, or with the present gasification or a combination thereof.

For these objectives, the above two working media 19 (i.e. 19.1 superheated CO₂ to supercritical CO₂ supraheating and/or 19.2 saturated steam from the adjacent plant, or the 53.1 supercritical CO₂ turbine and the 53.2 steam turbine for syngas recompression) are reheated by 20 downstream of reactor 40 (optionally downstream of the 49 in addition) before these streams are directed to the turbines, or are exported to other purposes as 21.1 and 21.2 respectively.

Finally, as the superheated CO₂ and superheated steam are not available at the start up period of the gasification and syngas downstream plants in the start-up time period, the import lines for supercritical CO₂ 22.1 and 22.2 for superheated steam (e.g. from the auxiliary gas turbine) are considered for running the turbines initially.

In addition, the steam flush lines 24 is considered to flush the natural gas lines and the gasifier in plant trip situations and during the shut-down period.
FIG. 14 Process heat and syngas generation by way of gasification of part of the CO₂ containing gaseous streams

### ELEMENTS IN THE FIG. 14

| | |
|---|---|
| 1 | CO2-Stream as the CO₂-enriched recirculated flue gas by the |
| | Advanced Combustion to the subcritical compression |
| 2 | Subcritical compressed CO2-Stream to supercritical |
| | compression |
| 3 | 3.1 Supercritical CO2-Stream from the Advanced Combustion |
| | to CO2-CC section |
| | 3.2 Subcritical compressed CO2-Stream for gasification |
| 4 | CO2-Sidestream, the oxygen-rich purge gas downstream of the |
| | CO2-CC's condenser(s) to preheater |
| 5 | Preheated CO2-Sidestream downstream of the expander turbine |
| | for gasification |
| 6 | CO2-Sidestream, the Regasified-CO2 stream from the CO₂ |
| | condenser(s) of the CO2-CC section |
| 7 | Collected O₂-containing CO2-Sidestreams to final preheating |
| | upstream of the gasifier |
| 8 | CO₂/O₂ conditioned gas blends to gasification |
| 9 | Natural gas upstream of the natural gas compressor |
| 10 | Hydrogen for hydrogenation of residual sulfur compounds in |
| | the compressed natural gas |
| 11 | H₂S containing compressed natural gas to desulfurization |
| 12 | Desulfurized compressed natural gas to preheater |
| 13 | Desulfurized compressed natural gas for gasification |
| | 13.1 Natural gas side stream for ignition (not depicted in the |
| | figure) |
| 14 | Desulfurized compressed natural gas for second stage |
| | gasification (in the autothermal gasification) |
| 15 | Preheated IP oxygen (downstream of the CO₂ condenser's and |
| | steam condenser's heat exchangers) for further preheating |
| 16 | Preheated IP oxygen for joining to the O₂-containing |
| | CO2-Stream for the gasification |
| | 16.1 Oxygen side stream for ignition (not depicted in the figure) |
| 17 | Hot IP syngas downstream of the gasification |
| 18 | HP/IP syngas to the adjacent plant for the final production |
| 19 | 19.1 Superheated CO₂ to supercritical CO₂ supraheating |
| | 19.2 Saturated steam from the adjacent plant (e.g. methanol, |
| | gasoline plant) |
| 20 | 20.1 Supraheated CO₂ to supercritical CO₂ turbines |
| | 20.2 Superheated steam to the steam turbines |
| 21 | 21.1 Supraheated supercritical CO₂ to other CO₂ turbine(s) e.g. |
| | driving generator for back-up AC or dispatch power |
| | 21.2 Superheated steam to other steam turbines, e.g. for driving |
| | generator for back-up AC or dispatch power |
| 22 | 22.1 Import superheated CO₂ for the supercritical CO₂ turbines |
| | for the start-up period |
| | 22.2 Import superheated steam for the steam turbines in the |
| | start-up period |
| 23 | Excess process water condensate from the gasification process, |
| | typically from 47 and 48 (not depicted in the FIG. 14) |
| 24 | Superheated steam line for gasification and flushing the system |
| | in shutdown mode |
| 25-30 | Not applicable |
| 31 | Subcritical flue gas compressor, i.e. CO₂-enriched CO2-Stream |
| 32 | Subcritical flue gas cooler upstream to the supercritical |
| | compressor |
| 33 | Supercritical compressor for mainstream of CO₂-enriched |
| | CO2-Stream to CO2-CC and eventually to the HPLTE-SG |
| 34 | Expander turbine for the oxygen-rich purge gas from the |
| | CO2-CC section (typically from 75 bar to 45 bar expansion) |
| | with generator |
| 35 | Natural gas compressor |
| 36 | 36.1 Supercritical CO₂ turbine |
| | 36.2 Steam turbine |
| 37 | Generator for power generation attached to the flue gas turbo |
| | machinery |
| 38 | 38.1 Supercritical CO₂ turbine |
| | 38.2 Steam turbine |
| 39 | Generator for power generation attached to the natural gas turbo |
| | machinery |
| 40 | Gasifier for process heat generation by use of gasification, i.e. |
| | for single stage gasification and/or two stage autothermal |
| | gasification |
| 41 | Exothermic gasification compartment of the reactor, typically |
| | the first stage of gasification in the autothermal gasification |
| 42 | Center tube of the (autothermal) gasifier with integrated static |
| | mixer section |
| 43 | Chamber for conditioned gases upstream of autothermal |
| | gasification |
| 44 | Endothermic steam reforming tubes with catalyst for (the |
| | autothermal) gasification |
| 45 | Collection chamber of the gasification reactor effluents |
| 46 | Process heat recovery downstream of the gasification reactor |
| | (i.e. preheater for CO₂/O₂ stream upstream of the gasification |
| | reactor) |
| 47 | Process heat recovery heat exchanger vs CO2-Sidestream, the |
| | oxygen-rich vent gas downstream of the CO2-CC section |
| 48 | Process heat recovery for oxygen vs process heat from syngas |
| 49 | Additional CO₂ superheater or steam superheater, not depicted |
| | in the FIG. 14. |
| 50 | Integrated process heat generator of the reactor for the |
| | superheated supercritical CO₂ or superheated steam |
| 51 | Natural gas hydrogenation reactor |
| 52 | Natural gas sulfur scavenger reactor |
| 53 | 53.1 Supercritical CO₂ turbine |
| | 53.2 Steam turbine |
| 54 | Syngas recompressor |
| 55 | Generator of the syngas turbo set machinery |

### ABBREVIATIONS, SPECIAL TECHNICAL EXPRESSION WITH THE ELABORATIONS

The present patent in continuation to U.S. Ser. No. 14/392,066 uses also some special technical expressions and terms. Other terms, not described below, have been marshaled in the parent patent, subpart for abbreviations, special technical expressions, and terms.

Advanced Combustion:

The processing pertaining to the CO2-Stream of the parent patent in the post-combustion carbon dioxide capture to accomplish the chemical oxidation of the fossil fuel in a way that the combustion no longer harnesses the air with the adverse concomitant nitrogen. This kind of processing implies in the present patent in continuation the use of recirculated clean flue gas with CO₂ as major constituent and oxygen in the post-combustion carbon dioxide capture. The related processing in the present patent comprises also the preparation and enhancement of CO₂ concentration in the post-combustion, which enables the extraction of a very CO₂-rich gas at very low gas flow rate for further processing.

Primary Carbon Ratio:

The U.S. Ser. No. 14/392,066 discloses a process, wherein the carbon constituent of the fossil fuel prior or post the combustion is regained as liquid carbon dioxide first, by then, that carbon dioxide is converted to cathodic syngas CO/2H₂ and anodic oxygen in high pressure low temperature electrochemical syngas generator HPLTE-SG.

The cathodic syngas as well as the anodic oxygen are further employed as condensing media for the closing of the First Bairamijamal Cycle (each stream repeatedly, vide FIGS. 5A and 5B), along the trajectory 6-7 in the FIG. 4A. These cathodic syngas and anodic oxygen are utilized as condensation media repeatedly, typically three or four times, to reclaim eventually the waste heat harnessed to back up the high-pressure electrolysis for the CO2-CC and CO2-PG.

Thus, the ratio of the mass flow rate of carbon in the circulating CO₂ in the First Bairamijamal Cycle to the mass flow rate of the carbon from the fossil fuel is of pivotal significance. With the consideration of three to four times expansions of the HPLTE-SG syngas (CO/2H₂) and oxygen (3/2 O₂) with their respective stochiometric ratios, the available primary carbon ratio can recover great part of the waste heat, yet not all of it (v.i. for secondary carbon ratio).

Since the mass flow rate of carbon of the fossil fuel to liquid carbon dioxide that is fed to HPLTE-SG relates to primary carbon mass stream, subsequently the generated cathodic syngas and anodic oxygen from the primary carbon are interlinked to the portion of carbon mass flow rate in the CO₂ cycle for the closing trajectory. For this reason, the more carbon rich the fossil fuel (like petcoke and coal) the higher waste heat can be effectively recovered, ergo the higher carbon constituent of the fuel, the higher gross efficiency of the process in both perspectives, i.e. in terms of plant gross thermal efficiency from the energy balance as well as in terms of revenue from the additional electricity to the grid, more significantly the revenue yielded from the high end final products.

Secondary Carbon Ratio:

The secondary carbon ratio is defined as the mol, rather mass flow rate of carbon in the First Bairamijamal Cycle to that of carbon monoxide of the cathodic syngas (CO/2H₂), then post water shift converter to carbon dioxide (CO₂/3H₂). The obtained liquid carbon dioxide downstream of the CO2-CC section can now contribute via additional units of HPLTE-SG to regain more cathodic syngas and anodic oxygen, which can now recover more waste heat. The additional stream of (CO₂/3H₂) downstream of water shift converters are termed secondary CO2-Stream that is subject to the third embodiment of present patent in continuation for its pre-combustion enhancement of carbon dioxide concentration of CO2-Stream (vide embodiment (III) and FIG. 13). Respectively the secondary syngas, yet more importantly the secondary oxygen for gas turbine plants are defined below.

The U.S. Ser. No. 14/392,066 discloses the high-pressure water-shift converter (elements 35 with related equipment) as well as the intermediary pressure water shift converter (elements 46 with related equipment) for total or partial conversion of the syngas from HPLTE-SG. By operation of the water shift converters, hydrogen (stream 70) and additional CO2-Stream (streams 37 and 48) is produced, from which liquid carbon dioxide is regained downstream of the CO2-CC sections as streams 38 and 49.

Either stream 38, and 49 can be redeployed to a HPLTE-SG that generates additional syngas and additional oxygen again, while both additional streams contributing to more waste heat recovery. This proceeding can be carried out only to the extent, as long as the waste heat recovery can economically back up the electrolysis and CO2-PG.

The deployment of any CO2-Stream(s) as presented in FIG. 3 streams 37 and 38, and it's further processing to regain liquid carbon dioxide therefrom, which can be redeployed with water to the HPLTE-SG for additional secondary syngas and secondary oxygen, is conducive to more waste heat recovery by the operation of the First Bairamijamal Cycle. The associated link between the carbon in the CO₂ mass flow rate of the First Bairamijamal to the carbon of the CO₂ mass balance in the redeployed liquid carbon dioxide to the HPLTE-SG is presented as the secondary carbon ratio. Both, the primary and secondary carbon ratio determine the scope of the economically reasonable waste heat recovery.

The additional hydrogen and oxygen generated essentially from the waste heat in addition to the primary hydrogen and oxygen are pivotal for supporting:
a) to the new generation of super-efficient hydrogen-based fossil power plants in two significant (FIG. 10).
b) running the fuel cell blocks for generation of DC current for the back-up of the remaining power for the electrolysis (vide description on the parent patent, and claim 60 therein)
c) delivering excess oxygen for the Advanced Combustion, if low carbon fuel like natural gas is utilized to run the fossil energy plant like in gas turbine power plants, vide infra

Primary CO2-Stream:

Relates to the carbon of the CO2-Stream that stems from the carbon constituent of the fuel

Secondary CO2-Stream:

With reference to FIG. 3, the one way is related to the conversion of the cathodic HP secondary syngas 33 in the HP water shift converter 35 to HP lean CO2-Stream 37. Thus, the present patent in continuation in the embodiment (III) pertains in one way to the pre-combustion carbon capture from this HP secondary CO2-Stream 37 that is routed to a CO2-CC section, wherefrom the liquid CO₂ is obtained again.

The second way is related to the conversion of the cathodic IP syngas (CO/2H₂) 43 downstream of syngas expander turbine 42 to the IP water shift converter 46, where an IP lean secondary CO2-Stream (CO₂/3H₂) 48 is prepared. Thus, the present patent in continuation in the embodiment (III) pertains in second way to the pre-combustion carbon capture from this IP CO2-Stream 48 that is routed to another CO2-CC section, wherefrom the secondary liquid CO₂ is also obtained again.

The enhancement of CO₂ concentration from either HP/IP lean secondary CO2-Stream to a CO₂-rich CO2-Stream appertains to this patent in continuation in the embodiment (III) with the FIG. 13 and the claims 63 and 64 related thereto.

The secondary oxygen, along with excess hydrogen, is also employed for the cell fuels feeding that generates DC current for the high-pressure electrolysis without any need for AC/DC converter. Through this backup DC line, the loss of electric energy by the AC/DC of ca. 12% to 15% can be reduced noticeably, while more AC can be dispatched to the grid. The secondary oxygen is further harnessed for production of some downstream final products e.g. nitric acid and other oxo-hydrocarbons.

Secondary Liquid CO₂

The collection of the both streams 38 and 49 above, constitutes the secondary liquid carbon dioxide. The generation of secondary liquid CO₂ and its re-electrolysis with water.

Secondary Cathodic Syngas

The secondary cathodic syngas CO/2H₂ is produced by re-electrolysis of the secondary liquid CO₂ with water in another HPLTE-SG units. Depending on remaining waste heat, the secondary syngas contributes to more to back-up the power for the electrolysis via the multi-stage syngas expander turbine and more hydrogen for super-efficient hydrogen-based fossil power plants.

Primary Anodic Oxygen:

Relates to the oxygen that is generated by the electrolysis from the primary CO2-Stream out of the carbon constituent of the fossil fuel. The ration of the primary carbon and primary oxygen is determined by the stochiometric ratio of 1 C to 3/2 O₂.

Secondary Anodic Oxygen:

The associated additional oxygen generated from the redeployed carbon dioxide back to electrolysis, presents the secondary oxygen.

The secondary oxygen, or simply secondary oxygen is of few paramount interests in the present patents. The parent patent presents a procedure to produce additional hydrogen and oxygen if the cathodic syngas CO/2H₂ undergoes a water shift converter with steam, whereas the CO₂/3H₂ is produced. This has been presented in the description and depicted in the FIG. 3 by two ways, namely one for lean HP CO2-Stream via 37, the other way for IP lean CO2-Stream via 48.

The secondary anodic oxygen enables the present process to compensate the oxygen deficit of 1 C to ½ O₂ in the mass balance if low-carbon fuel like natural gas is combusted, e.g. in CHP or gas turbine, for the CO2-Stream. The additional oxygen from this stream is termed secondary oxygen (vide table Advanced Combustion applied for an existing gas turbine).

Auxiliary CO2-Streams

The auxiliary liquid and gaseous carbon dioxide streams are considered in the parent, as well in the patent in continuation for some purpose. In the followings, few auxiliary CO2-Streams are disclosed with short elaboration related thereto.

Auxiliary Injection CO₂ for De-Superheating:

There are number of regenerative heat exchangers considered in the First Bairamijamal Cycle of the parent patent in position to exchange the residue heat of supercritical CO₂ downstream of the CO₂ turbine along the trajectory 6-7 in the FIG. 4A. These heat exchangers are in place to exchange the hot end of the CO₂ cycle past the turbine in point 6. At the other side of these heat exchangers, there is the upcoming preheated cold end of the cycle. This takes place first by integration of the waste heat from the point 2 over the critical point 3 to 4, wherein waste heat is recovered up to an economically reasonable point between the trajectory 3 to 4. Along this trajectory post the point 3 to 4, those said regenerative heat exchangers operate in counterflow.

Respectively, the last regenerative heat exchangers operate somewhere between ca. 160 Deg F to 110 Deg F. From there on to the critical zone around the 88 Deg F, the injection of liquid auxiliary carbon dioxide is considered into the supercritical carbon dioxide as a measure for de-superheating. This position of the de-superheating CO₂ is considered as Auxiliary Injection CO₂ for De-superheating. The auxiliary injection CO₂ is resupplied by secondary liquid CO₂, outlined above.

The injection CO₂ serves also as the “make-up CO₂” to compensate the regular discharge of CO₂ from the cycle. The other usage of injection of liquid carbon dioxide is for de-superheating purposes, as indicated in FIG. 12, steam 13.

Auxiliary CO₂ in ACU:

The parent patent considers the ACU, Auxiliary Cooling Unit for the supercritical (as well as subcritical condensation upon certain conditions). If the CO₂ is utilized as cooling and chiller media, the continuous resupply of that CO₂ is also to be provided via secondary CO2-Stream over the secondary liquid CO₂.

Regasified-CO2:

The liquid CO₂ that has been utilized in the ACU as condensing media for the condensation of the CO₂ out of the flue gas to the CO2-CC section. Thus, the Regasified-CO2 has been obtained by partial release of the liquid CO₂ from the supercritical CO₂ pressure down at ca. 32 Deg C. to ca. 45 bar at ca. 10 Deg C. This regasified CO₂ stream is then harnessed for the gasification reaction rather than to more costly recompression and rerouting back to the CO2-CC section. Hence this, it is referred to as the Regasified-CO2.

CO2-Sidestreams:

The CO₂ containing side-streams, which are either not economically advantageous to be rerouted back to the core line of the processing in the CO2-CC cooling and condensation (e.g. the Regasified-CO2 stream) or are not suitable from process economy are collectively referred to CO2-Sidestreams.

For reasons of overall process economy, i.e. scope of the investment for the equipment as well the operation costs, these CO2-Sidestreams are preferably deployed for process heat generation in the gasification. The present process utilizes these side streams for the gasification unit in order to generate more valuable syngas and also generation of additional process heat that eventually contributes to more DC back-up power supply to the HPLTE-SG units. The other reason is grounded on process media suitability, for instance the oxygen-rich CO2-containing purge gas at higher pressure level, which are suitable for syngas generation by use of gasification.

The integration of CO2-Sidestreams buttresses the persuasion of the Zero CO₂ Fossil Energy principal while considering the best available processing from technical and economy feasibility.

The ratio of the CO2-Sidestreams, as well the portion of the recirculated CO₂-rich flue gas are defined by the molar ratio to the origin process media, whence they are derived.

Gas Turbine Trippel-Combined Cycle:

A new generation of gas turbine combined cycle, whereas now the Brayton Cycle of the gas turbine with the new Advanced Combustion is combined with a heat recovery section that integrates the current water-steam cycle of the Rankin Cycle together with the First Bairamijamal Cycle operating with the carbon dioxide as working fluid. Based on estimates, the new gas turbine triple combined cycle can reach out to a gross efficiency of 80% to 85% with a GE 9HA Class gas turbine.

Compared with the most advanced gas turbine combined cycle with HRSG based on 9HA Class to 62.3%, the present patent in continuation presents a quantum leap in gross efficiency in the gas turbine power generation.

Dual-Combined Cycle Power Plants

The application of the Advanced Combustion that is integrated in a new fossil fuel fired power plant with coal, crude oil, biomass, waste carbonaceous material and the natural gas, which heat recovery section is consisting of a water-steam cycle according to the classic Rankin Cycle and includes section for the heat recovery with carbon dioxide as the working fluid according to the First Bairamijamal Cycle.

### SOME SELECTED REFERENCES

[1] Utilization of very dilute carbon dioxide from the air at 0.041 Vol %, absorption by use of amines, desorption and conversion of CO₂ to fuel https://energyfactor.exxonmobil.com/news/global-thermostat/ https://globalthermostat.com/dr-peter-eisenberqer/
[2] Harnessing carbon dioxide from the air at 0.041 Vol % by Carbon Engineering Inc., Canada https://carbonenqineering.com/
[3] Utilization of geothermal carbon dioxide at high concentration of ca. 95 Vol % for sustainable methanol of the Carbon Recycling International, commonly known as George Olah plant https://en.wikipedia.orq/wiki/Carbon_Recycling_International
[4] EIA report for electric power generation in 2019 https://www.eia.gov/tools/faqs/faq.php?id=427&t=3

61. The process for post-combustion carbon dioxide capture and re-use of that captured liquid carbon dioxide to syngas and oxygen out of a CO₂-lean CO2-Stream (i.e. in this case the flue gas of a fossil fueled power plant) according to the claims of 1, 2, 3, 4, 6, 8, 11 and 20, wherein the CO2-Stream of present process invention is further characterized by the enhancement in CO₂ concentration through the recirculation of a portion of the CO₂-rich CO2-Stream back to the combustion section and/or the HRSG section of the plant at one side, while the other portion of that said CO2-Stream is discharged to further processing for CO₂ condensation from that CO₂-rich CO2-Stream in the CO2-CC section, whereas the circulated CO2-Stream and respectively it's discharge portion upstream of the CO2-CC section reaches a CO₂ concentration between 60% to 95% by volume with the remaining portion of that said CO02-stream consisting utmost of oxygen

62. The process for post-combustion carbon dioxide capture and re-use of that captured liquid carbon dioxide to syngas and oxygen out of a CO₂-lean CO2-Stream (i.e. in this case the flue gas of a fossil power generation plant) according to the claims 1, 2, 3, 4, 6, 8, 11, 20 and 61, wherein that said CO2-Stream is recirculated and further distinguished by the addition of pure oxygen from the HPLTE-SG section of this process to the extent of 25% to 40% oxygen by volume in the recirculated CO2-Stream (also referred to flue gas oxy-fueling), in order to replace the nitrogen-rich intake air for the combustion entirely, while keeping a residual oxygen in the flue gas post the combustion and HRSG between 5% to 25% by volume.

63. The process for pre-combustion carbon dioxide capture and re-use of that captured liquid carbon according to the parent's patent claims 1, 2, 3, 4, 6, 8, 11, 20, in pursuant to the claims 61 and 62 forms the CO2-Stream according to this patent in continuation, in which that said CO2-Stream itself can originate from a CO₂-water electrolyte fed to a low pressure-, intermediary pressure-, specifically that CO2-Stream can originate from a high pressure low temperature electrochemical electrolysis, referred to as HPLTE-SG, wherein it's electrolyte has been consisting of liquid CO₂ and water- and this electrolyte is dissociated in the HPLTE-SG to that said CO2-Stream as the cathodic syngas in the composition CO/2H₂ first, by then that said CO2-Stream of CO/2H₂, either totally or in part further processed to water shift converter(s), whence the syngas in composition CO/3H₂ is obtained, wherein this syngas in composition of CO/3H₂ with the concomitant CO₂ post the water shift converter(s) presents this said pre-combustion CO2-Stream, which is specifically characterized in the present patent in continuation with the following peculiar features:
(i) the pre-combustion of this said CO2-Stream stems from LP-, IP, more specifically from HPLTE-SG, the high-pressure low temperature electrochemical syngas generator(s) post the water shift converter(s)
(ii) this CO2-Stream is processed through a CO₂ absorption/desorption process (i.e. use of abrorbens as Selexol, Rectisol, Benfield or any other) for separation of CO₂ from the CO₂/3H₂ CO2-Stream prior to any combustion,
(iii) whence a high concentrated CO₂-rich CO2-Stream obtained from the desorption above, and
(iv) the obtained CO₂-rich CO2-Stream is purified by intermittently operating scavenger adsorber column, wherein the traces of absorbens (e.g. Selexol, Rectisol, etc.) and water are removed to ppb concentration, thus
(v) that CO₂-rich CO2-Stream can be routed to the parent patent's CO2-CC section for cooling and separation of carbon dioxide via condensation will be performed preferably in supercritical pressure above the 1070 psi,
(vi) whereas the carbon monoxide purge gas downstream of the CO2-CC section is recompressed and rerouted back to the main gas stream of HPLTE-SG, upstream to the water shift converters.

64. The process for pre-combustion carbon dioxide capture and re-use of that captured liquid carbon from the CO2-Stream according to the parent patent, in which that said CO2-Stream itself originates from a LP-, IP, or HP-gasification process according to the according to the claims 1, 2, 3, 4, 6, 8, 11, 20, 61, 62 and 63, wherein the CO₂/CO/H₂ gas in any ratio can be processed in total or in part through the water shift converters to increase the hydrogen constituent, which is specifically characterized in the present patent in continuation with the following peculiar features:
(i) The pre-combustion CO2-Stream stems from LP-, IP, more specifically from HP-gasification process
(ii) This CO2-Stream is processed through a CO₂ absorption/desorption process (i.e. use of absorbens as Selexol, Rectisol, Benfield or any other) for separation of CO₂ from the CO₂/CO/H₂ containing CO2-Stream prior to any combustion,
(iii) whence a high concentrated CO₂-rich CO2-Stream obtained from the desorption above, and
(iv) the obtained CO₂-rich CO2-Stream is purified by intermittently operating scavenger adsorber column, wherein the traces of absorbens (e.g. Selexol, Rectisol, etc.) and water are removed to ppb concentration, thus
(v) CO₂-rich CO2-Stream can be routed to the parent patent's CO2-CC section for cooling and separation of carbon dioxide via condensation will be performed preferably in supercritical pressure above the 1070 psi,
(vi) Whereas the carbon monoxide purge gas downstream of the CO2-CC section is recompressed and rerouted back to the main gas stream of HPLTE-SG, upstream to the water shift converters

65. The process for post-combustion carbon dioxide capture and re-use of the captured liquid carbon dioxide to syngas and oxygen out of a CO₂-lean CO2-Stream is distinguished, wherein the carbon monoxide traces trace in the recirculated flue gas will be oxidized by use of catalytic reaction with the concomitant oxygen post flue gas oxy-fueling and conditioning of the recirculated flue gas by preheating, wherein preferably hydrogen-oxygen torch(es) is employed for the reheating.

66. The process for post-combustion carbon dioxide capture and re-use of that captured carbon to syngas and oxygen out of a CO₂-lean CO2-Stream is distinguished to perform Zero-CO₂-Emission Fossil Energy, that implies the capture and recovery of any low flow rate or lean CO2-Stream by use of a CO₂ absorber that absorbs the residual carbon dioxide with water, particularly from the purge gas of CO2-CC section, i.e. the oxygen-rich purge gas and recovers that said absorbed CO₂ by desorption process, which takes place preferably in situ in the scrubber of the parent patent

67. The process for post-combustion carbon capture and re-use of that captured liquid carbon to syngas and oxygen out of a CO₂-lean CO2-Stream is distinguished, wherein the oxygen-rich purge gas from the CO2-CC section post the CO₂ absorber will be dehydrated and undergoes partial expansion that delivers liquid oxygen LOX and gaseous oxygen GOX out of that oxygen-rich gas with the none-condensing gases.

68. The process for post-combustion carbon capture and re-use of that captured liquid carbon dioxide according to the claims 1, 2, 3, 4, 6, 8, 11, 20, 61, 62, 63 and 64, whereas the Advanced Combustion is integrated in a new fossil fuel fired power plant—i.e. coal, crude oil, biomass, waste carbonaceous material and natural gas—is implemented in a Dual-Combined Cycle, wherein the heat recovery is consisting of combination of classic Rankin Cycle with steam as working fluid with the First Bairamijamal Cycle with the carbon dioxide as working fluid that leads to a gross thermal efficiency in the margin of 65% to 75%.

69. The process for post-combustion carbon capture and re-use of that captured liquid carbon dioxide, according to the claims 1, 2, 3, 4, 6, 8, 11, 20, 61, 62, 63 and 64, whereas the Advanced Combustion is implemented in a new gas turbine Triple-Combined Cycle implemented, wherein the (i) classic Brayton Cycle; (ii) with the HERSG of the classic Rankin Cycle; and (iii) the First Bairamijamal Cycle are combined together that leads to a gross thermal efficiency in the margin of 80% to 85%.

70. Process for the preparation of CO₂-enriched CO2-Stream at high CO₂ concentration with its constituent—i.e. utmost oxygen—obtained from the flue gas of fossil fuels according to the claims 61 to 66, is harnessed for DC back-up power generation and syngas generation, wherein the concentrated CO2-Stream is routed in one part to a gasification process for generation of additional process heat, which in turn is either integrated in the CO2-HR and CO2-PG of the First Thermodynamic Cycle with the supercritical CO₂ turbine(s) and/or is carried out for generation of superheated steam with steam turbine(s), whereas either working media is eventually utilized for generation of primary AC current in order to back up the HPLTE-SG electrolysis with additional DC power supply post the AC/DC converter of the process.

71. Process according to the claims 61 to 66, and 70, whereas other CO2-Sidestreams are as well routed to that said part of the flue gas to the gasification for additional process heat generation in order to reduce the extent of processing work and equipment with those side-streams, whereas the CO2-Sidestreams are specifically; (i) the re-gasified pure CO₂ that has been utilized as condensing media for the condensation of the CO2-Stream in the CO2-CC section by partial pressure relief of the liquid carbon dioxide into lower pressure level of ca. 45 bar, which typically consists of about 5% of the circulated flue gas' CO₂ to the CO2-CC section, and (ii) the oxygen-rich CO₂ containing vent gas downstream of the CO2-CC condenser(s) with over 90 Vol % oxygen and to lesser portion of CO₂ constituent at ca. 75 bar pressure, which typically constitutes nearly 10% of the origin CO₂-enriched CO2-Stream of the Advanced Combustion to the CO2-CC section.

72. Process according to the claims 61 to 66, then 70, and 71 distinguished in generation of additional process heat, whereas the gasification of that said CO2-Sidestreams with portion of that CO₂-enriched CO2-Stream is carried out with the anodic oxygen obtained from the HPLTE-SG and the natural gas preferably a single-stage gasification, more preferably to a two-stage autothermal catalytic gasification (also referred to as bi-reforming).

73. Process according to the claims 61 to 66, then 70 to 72 is further characterized that the process heat generation in the gasification of that said CO02-streams with oxygen and natural gas is integrated in the overall heat recovery CO02-HR and CO2-PG with the supercritical CO2-turbine(s), syngas turbine(s), oxygen turbine(s) and the steam turbine(s) for DC back-up power, yet then as such, it comprises also the superheating of saturated steam obtained in the adjacent downstream production plant, where the conversion of the syngas takes place to produce the final products, i.e. methanol, transportation gasoline, aviation fuel, ethanol, ammonia, and other final products.

74. Process according to the claim 70, 71, 72 and 73, wherein that portion of the primary CO₂-enriched oxygen-containing CO2-Stream (i.e. the circulated primary flue gas from the Advanced Combustion, aka termed the flue gas oxy-fueling; i.e. according to figure #14, the stream #1) with the other CO2-Sidestreams (according to figure #14, i.e. the streams #3.2, #4 and #6) are fed singularly or jointly to that said gasification, comprises 1% to 45% of the primary carbon mol stream as carbon dioxide over the circulated flue gas (which is the stream #1), more preferably that the CO₂ mol stream to the gasification is carried out between 15% to 25% of the primary mol stream of carbon obtained post combustion as carbon dioxide obtained from the primary carbon mol stream over the circulated flue gas.

75. Process according to the claims 70, and 72 characterized that the extent of the secondary natural gas injection into the second stage of the catalytic autothermal gasification is specified in ratio to the generated water steam in the first stage of the gasification in a way, that mol ratio of the water steam of the first stage to the secondary natural gas into the second stage stands in the mol ratio of (1.75-3.0) water to 1 natural gas, more preferably between 2.5 mol water to 1 mol natural gas.

76. Process according to the claims 60 to 66, and then 70 and 71 characterized that the low temperature oxygen-rich CO2-Sidestream from the purge gas downstream of the CO2-CC condenser(s) at ca. 75 bar is integrated in the CO2-HR and CO2-PG by preheating from waste heat and process heat, then routed to a turbine with the attached generator to generate additional AC power, before it is added to that portion of the CO2-Stream to the gasification.
